SEQUENCE LISTING (8 sequences)

SEQ ID NO: 1 - DNA, 2466 bp, Homo sapiens
gtcgagagcggagcagactggttaaggacatgagacccagaggaataacctggtgatggacagaagatggaaaaatttcttacatgaatgaaaagcctctggtatgtcagaaccatgctgtcttccatgagacttcctttgtgaagagcatccatttaaaagacttttatgaatacatggtttcaatcaagtccccagagaacacatttgtcttctgagctgctggcagttttgagaatctgatgacctccgaggggaccctgcactcagccatcaaagtgttcctgcccctctggacactcataattcaatggtagatgcaggtggagttgagaacatcacccagcttccccaggagcttcctcagatgatggctgcagcagccgatggtttggggagtatagcgatagacacgacccagctcaacatgtccgtgacagatcccacagcctgggctacagccatgaataacctgggcatggttcccgtagggttgcctggacagcagctcgtgtctgactcaatctgtgtcccaggctttgatccaagcctcaacatgatgactggaatcacccccattaacccaatgataccaggccttggactggtacctcccccaccaccaacagaagtggctgttgtcaaagaaataatccactgcaaaagctgtactctttttcctcaaaatccaaatcttccacctccttccacaagagaacgacctcctgggtgtaagaccgtgtttgtcggaggattaccagaaaatgctactgaggaaattattcaagaagtctttgaacagtgcggtgatattacagcaattcggaaaagcaagaagaatttttgtcacattcgctttgcagaggaattcatggttgataaagccatttacctttctggttataggatgcgattagggtctagcaccgacaaaaaggattcaggccgccttcatgtggactttgcccaggccagggatgacttctatgagtgggaatgcaagcagaggatgcgtgcccgggaggagcggcaccggcgcaagctggaggaggaccggctcaggcccccatccccgcctgccataatgcactactcggagcacgaagccgctctgctggctgaaaagctgaaagatgatagcaagttttcagaggctatcacagtgctgctttcctggattgaacgaggggaagtgaatcggcgctctgcaaaccagttctattccatggtgcagtcggccaacagccacgtccgccggctaatgaatgaaaaagccacccatgagcaagagatggaggaagccaaggagaattttaaaaatgccttaactgggattctcactcaatttgagcagattgtggccgttttcaacgcttctaccagacaaaaagcttgggaccatttctcgaaagcccagcgcaagaacatagacatttggcgaaagcattctgaggagctccggaatgctcaaagtgagcagctcatgggcatccgccgcgaagaagaaatggaaatgtctgatgatgagaactgtgacagccctacaaagaaaatgagagtcgatgaatcagccctggctgcccaggcctacgctctgaaagaggagaatgacagtctccgctggcagctggatgcctacaggaatgaggtggagctgctgaaacaagaaaaagaacagcttttccgaacagaagaaaacctcaccaaggaccagcaactgcagtttctgcagcaaaccatgcaaggcatgcagcagcaattgctaaccatccaggaggagttaaacaacaaaaagtcagaattggaacaagcaaaggaagagcagtcccatacacaagcgttactaaaagtcctgcaggaacaattaaaaggtaccaaggaattggtcgagaccaatggccacagccatgaggattcaaatgaaatcaatgtgttgacagttgcattagtcaaccaagaccgagagaacaatattgagaaaagaagccaaggcttaaaatcagagaaagaagctctgctaataggtatcatatcaacgtttcttcacgtccatccttttggagccaacatagaatatctttggtcatacatgcagcagctggactccaagatatctgcaaatgaaatagaaatgcttttgatgaggctgccacgcatgttcaaacaggaattcacgggtgtgggagccacgctggaaaaaagatggaagttgtgtgcctttgaaggaattaaaactacctaactgcgaagagcaaagcatctctggaaatgaaaccatgtgaacctggccagggcggtgcgacggggaagcaggaggtgtggggttggtcccgcacgcaacctttgtggagccatcgaagcctgcctttagttatatctgtggcgttctcttgtaagtggaaatgtaattgtgtaccagtttcttaaaataaacaaagcttcatactgtgaaaaaaaaaaaaaaaa

SEQ ID NO: 2 - DNA, 3405 bp, Saccharomyces cerevisiae
atgtcaaattccaattcaaaaaaacctgttgcgaattacgcataccgtcaacagcaagattataatgggatgaacgccatggtgggaaatccaatgatgtatcatcctgttgatttcgtaaacggtgccggccaatatggtccttctcaacacccagcatattacaccaactcaccattacctaatattccaccaacaccttttgatactgcatacggtgccagtctttttccatctcacctattgatgggatctccatttgtttcctcgcctaacatgcaaagtggctataattctgcaagatcatctaatctcaaaagaaaggcttattcgaggcccgtttctaatcataacggttacaacggaaacagtaacagtaaccaaaacaatactaataacggaatggtaacaccctcgaactattatagaatggggagaaattctttctcgaggaacaacaacagtacgaggaatgttactcacaacaacaataaggggtgcgacacccggaacaacagtggaagaagaacattcgcaaggaataacattttcgacgacatacttccagaaatgcttttacaaagacccttttgtattaattacaaggttctaccgactggtgatgatgcttatagaactaggtcgctgcttattgaaaatgtggatcattctattgatttacactctatagtcaaaaattttgtcaaatccaatactctggaaagtgcctacttaattgaaggagggaagagcgatgattcaaaagatgtagagactaaaaatctatccattctaataagctttctaaccaaaggcgactgtttgaacttttacaataacattttacaaaggttatctgaattcaaaacatttttgaaatctgaagcactaaatctgaaatttgtttgtctaaactatgatcccaaatgcctccctacctttattgagagtgaagcgttaacagaaaacgctgaagaagccgacattaccaatggttcaacaatgatcagtgcttcgctgcatcataatatagcgaacaaagatgctacaaggtcgattataattgaattcaaaagccccgtggagaaaagcgatttattcaaaaagaaattacaatttttggacaggtcaaaaaacaaaagatacattttggaatctatcgatttagtgaacacagatgtaccttccaatcaatttcctgaaaattacgccgttttaacctttttgaatatctctatggctattgaagttctcgattatttgaaaaaatactccaaaaacttaggcatttctaaatgtttttacgtatccctagccccgttggtagttagctcagccagatcttcggttgctaatatttacgagggtaaaacgagcacacatcgtttatcggtgccttctgttactgctgggaacaataacgatagcaacaacaacggaaacaataataaaagtaatatgagtggtatcaccacactcaataataatagtagtattggtgtttccgtatacggtcactctaatatgagtttaaccagtctgtcatcatctgtatctttaaatgaagagattgatatgcttgcgacgaaacttcaaggggtagaacttgatgggacttatttagaaatcaactatcgcgactatcaaacaccaactattgaagaacactctactcacttgagcaatgtcaaaatttcaaagacaacagaaaactctaggcaattttctcaagatatcccatcacctttgccattaaatgaacatatgtttatgaatgattctaatcagtccaatggagcgataatacctcaacagttaatagccacaccttctccggtatcccccaatttgcaaatgaatcaaagggtgttgccgaatccaataactcaaagtttggagcaaaatttcaacgtttcggccaaagtggcatcatccatgggttcagacataggcaatagaacaatttatataggaaacattaatccaagatcaaaggcggaggatatctgtaatgtcgtgcgtggaggaatccttcagagcattaagtacataccggagaagaagatatgctttgttactttcatcgaagctccatccgctgtacagttctatgcaaattcatttattgatccaatagtgttacatgggaatatgttgagagttggatgggggcattattctggtccattaccgaaattaatctcgttggctgttacgatcggggcaagtaggaatgtctacgtaagcctaccagagtttgcatttaaggaaaagtttattcacgaccctcagtacaaaaaattgcatgaaacactatctttaccagatgcggaacaactaagagaggactttagtacctatggtgatattgagcagattaactatttgagtgatagccattgctgttggataaatttcatgaatatatcttctgcgattagtcttgttgaagaaatgaataaggaatccacagtacaaaatgaatccggtgaagtgacgcttaaaagggcgactgaggaaaaattcggtggccgttataagggccttctgataaactacggcaaagatcgttgtggcaatataaacaaaaatttgatagcaggtaaaaattcaagattctataagaaagttaaaagaccgagctataatatacgtttgagtaagttggaagaaaagaggaggcagaatgaaatcgatgaaaaggaaaaggcttttgataaacccttgaacttggaatccctaggaattagtctggacgcacataaggacaacggcggtggtgaaacaggaactgcaaataatactgggcatgaaaatgaaagtgaactagaggctgaaaatgaaaacggtaatgagactggaagtttcggtggactgggtctcgctgtggcgagctctgacgtcaaacgtgcgacatcggatgaaactgattatgaagatatatttaacaagtcatcgggatcttccgactcgtcatcagacgtcgaggtcattatgcactccccgagcgatcctgaatacgctttaaaatcacaaactctaagaagctcgagtcagaccgtcattaatagtaagagaccagtaaagatagaagacgaggaagaagccgtaggaatgtcacagctcaattataggtcgtcattaagacaagctcctccaagagctccctcaactttgtcatataatcactcgaagaacaacgaaacgccaatgcaagatattttcacaaatggcgaaacagcaaataacagaaagaagaagagaggatcttttgcaaggcatagaacgataccaggatctgacgtcatggcccaataccttgcacaagtgcaacattcgacatttatgtatgcagccaatattttgggcgcctctgcggaagacaacacgcatcctgacgagtag

SEQ ID NO: 3 - DNA, 3447 bp, Arabidopsis thaliana
tttttattttacttttcaacaagcgaagcgaaccctcgatctcaaggcgaaagcagatttcgtagcttccatggcgagaaattcgaattccgatgaggcattctcgtcagaggaggaagaagagcgggttaaggataatgaagaagaagatgaggaggagctcgaggctgttgctcgttcttctggctccgacgatgacgaagtagtcgccgccgacgaatccccagtctccgacggagaggctgctcccgtagaagatgattacgaggacgaagaagatgaggaaaaagctgaaatcagcaaacgtgagaaagccagacttaaagagatgcagaagttgaagaagcagaagattcaagagatgctggaatcacaaaatgcttccattgacgcggatatgaacaacaagggaaaagggagactgaagtatctcctgcagcaaaccgagttatttgcacactttgctaaaggtgatgcatcttcttctcagaagaaggctaaaggaaggggtcgtcatgcttccaaaataactgaagaggaggaagacgaagagtatctaaaggaagaagaggatggcttaactggatctggaaacacacggttactcacacagccctcttgtattcaagggaagatgagagattaccaattagctggtttaaactggctcattcggctgtatgagaatggcataaatggaattcttgctgatgaaatgggtctggggaagacgcttcaaacaatttctttgttggcataccttcatgaatacaggggaatcaatggtccccatatggtggttgctccaaaatcaacacttggtaattggatgaacgaaattcgccggttttgtcctgtcctacgtgctgtgaagttccttggtaatcctgaggagaggagacatattcgagacgacctgctagttgctgggaaatttgatatctgcgtcacaagctttgagatggccatcaaagagaagacagcacttcgtcggtttagctggcgttatattatcatcgatgaagcgcatagaatcaagaacgagaattcactcctttctaaaaccatgagactttttagcaccaattatcggcttcttatcacgggaaccccccttcagaataatctccatgaactgtgggctcttctaaattttcttctgcctgagatttttagttcagcagagacttttgatgaatggtttcaaatttctggtgagaatgaccagcaagaagttgttcaacaacttcacaaggttcttcgaccatttcttcttcgaagactaaagtcagatgttgagaaaggtttgccaccgaagaaggagaccatacttaaagttggtatgtctcagatgcaaaagcaatactacaaggctttactgcagaaggatcttgaagcggttaatgctggtggagaacgcaaacgtctgctaaacattgcaatgcaactgcgtaaatgctgcaatcacccttatctcttccagggtgcagaacctggtcccccatataccacaggagatcaccttataacaaatgcgggtaagatggttctcttggataaattgcttcctaagttgaaagaacgtgattcaagggttctgatattttctcagatgacaagacttttggatattcttgaggactatttaatgtatcgtggttacctatattgccgcattgatggaaacactggtggtgacgaacgagatgcctccatagaagcctacaacaagccaggaagtgagaaatttgttttcttgttatctaccagagctggagggcttggtatcaatcttgctactgcagatgttgttatcctgtacgatagtgattggaacccacaagtcgacttgcaagctcaggatcgtgcccatagaattggtcaaaaaaaagaagttcaagtgtttcgattctgcactgagtctgctattgaggagaaagtgattgaaagagcttacaagaagttagcacttgatgctctggttattcaacaagggagattggcagaacagaaaactgtcaataaggatgagttgcttcaaatggtaagatatggtgctgagatggtgttcagttctaaagatagcacaatcacagacgaggatattgatagaatcattgccaaaggagaagaggcaacagctgaacttgatgccaagatgaagaaattcacagaagatgcgatacagtttaaaatggatgacagtgctgacttctatgattttgatgatgacaataaggatgaaaacaagctcgattttaagaagattgtgagcgacaactggaatgatccccccaaacgggagagaaagcgcaactactctgaatctgaatactttaagcaaacattgcggcaaggtgctccagctaaacctaaagagcctagaattccgcgcatgccccagttgcacgatttccagttctttaacattcagagattgaccgagttgtatgaaaaggaagtacgctatctcatgcaaacacatcagaaaaatcagttgaaagacacaattgatgttgaagaaccagaaggtggagatcccttaactgctgaagaagtagaagaaaaggagctattattggaggagggtttctcaacatggagtagaagagatttcaatactttcctcagggcatgtgagaagtatggccgcaacgacataaaaagcattgcctctgagatggaagggaaaacagaggaagaagttgaaagatatgccaaagtattcaaagagcgatacaaggagctgaacgactatgatagaatcattaagaatattgagaggggagaggcaaggatctctaggaaagacgaaatcatgaaagccatagggaagaagctggatcgctacagaaacccttggctggaactgaagattcaatatggtcagaacaaaggaaagttgtacaatgaagagtgcgaccgtttcatgatctgcatgatccacaaacttgggtatgggaattgggatgagctgaaggcagcattcaggacatcgcctttgttcaggttcgactggtttgtgaaatctcgcacgagtcaggaacttgcaagaagatgcgacaccctgattcgactgatcgagaaagagaaccaggagtttgatgaaagagagaggcaagcccgtaaagagaagaagctcgcaaagagtgcaacaccatcaaaacgacctttaggaagacaagccagtgagagtccttcatcgacgaagaagcgaaagcacctgtcgatgagatgagattatgtgtttattgatttgttgagtctttgttgttacatatagttaagctaatgtcaaaaacatagcaaatggatttcattctgctttcgtgaatgtttgtcggctggtttgacttggatttaatgaaattacttaattataattttaaagaaactgaacaaaaagatttgaggccattggatggcaacttaggccgatgcttaagc

SEQ ID NO: 4 - DNA, 642 bp, Prunus persicaria
ccccaggcaacgatgattttccctatcttgttcacattttttctcctcctctcctcctccaatgcggctgtgcaagacttctgtgttgcagacttagcagctcctgaaggccctgcaggcttctcttgcaaaaagcctgcaagtgttaaagtaaatgattttgtgttctcgggcctgggcattgctggtaacaccagtaacatcatcaaagctgcagtcacccctgcatttgttgctcagtttcctggtgtgaatggcctcggcatttccatcgcccgtctagacttggcggttggcggagttgtcccatttcacacacaccctggagcttcagaagtcctaattgtcgcccaagggacaatctgcgccgggtttgttgcctcagataacacaccttatctgcaaactcttgagaagggtgacattatggttttcccacaggggctgttgcacttccaagtcaatggaggtgaggctccagcccttgcatttgctagcttcgggagtgcaagcccgggtctccaaattctggactttgctttgttcaaaaacgatttgcctaccgaagtaatagctcagactactttccttgatgctgctcagataaagaaactgaagggtgttcttggtggtactaattaa

SEQ ID NO: 5 - protein, 1055 aa, Arabidopsis lyrata
MARNSNSDEAFSSEEEEERVKDNEEEDEEELEAVARSSGSDDDEVVAADESPVSDGEAAPVEDDYEDEEDEEKAEISKREKARLKEMQKLKKQKIQEMLESQNASIDADMNNKGKGRLKYLLQQTELFAHFAKGDASSSQKKAKGRGRHASKITEEEEDEEYLKEEEDGLTGSGNTRLLTQPSCIQGKMRDYQLAGLNWLIRLYENGINGILADEMGLGKTLQTISLLAYLHEYRGINGPHMVVAPKSTLGNWMNEIRRFCPVLRAVKFLGNPEERRHIRDDLLVAGKFDICVTSFEMAIKEKTALRRFSWRYIIIDEAHRIKNENSLLSKTMRLFSTNYRLLITGTPLQNNLHELWALLNFLLPEIFSSAETFDEWFQISGENDQQEVVQQLHKVLRPFLLRRLKSDVEKGLPPKKETILKVGMSQMQKQYYKALLQKDLEAVNAGGERKRLLNIAMQLRKCCNHPYLFQGAEPGPPYTTGDHLITNAGKMVLLDKLLPKLKERDSRVLIFSQMTRLLDILEDYLMYRGYLYCRIDGNTGGDERDASIEAYNKPGSEKFVFLLSTRAGGLGINLATADVVILYDSDWNPQVDLQAQDRAHRIGQKKEVQVFRFCTESAIEEKVIERAYKKLALDALVIQQGRLAEQKTVNKDELLQMVRYGAEMVFSSKDSTITDEDIDRIIAKGEEATAELDAKMKKFTEDAIQFKMDDSADFYDFDDDNKDENKLDFKKIVSDNWNDPPKRERKRNYSESEYFKQTLRQGAPAKPKEPRIPRMPQLHDFQFFNIQRLTELYEKEVRYLMQTHQKNQLKDTIDVEEPEGGDPLTAEEVEEKELLLEEGFSTWSRRDFNTFLRACEKYGRNDIKSIASEMEGKTEEEVERYAKVFKERYKELNDYDRIIKNIERGEARISRKDEIMKAIGKKLDRYRNPWLELKIQYGQNKGKLYNEECDRFMICMIHKLGYGNWDELKAAFRTSPLFRFDWFVKSRTSQELARRCDTLIRLIEKENQEFDERERQARKEKKLAKSATPSKRPLGRQASESPSSTKKRKHLSMR

SEQ ID NO: 6 - protein, 168 aa, Saccharomyces cerevisiae
CCWINFMNISSAISLVEEMNKESTVQNESGEVTLKRATEEKFGGRYKGLLINYGKDRCGNINKNLIAGKNSRFYKKVKRPSYNIRLSKLEEKRRQNEIDKKEKAFDKPLNLESLGISLDAHKDNGGGETGTANNTGHENESELEAENENGNETGSFGGLGLAVASSDV

SEQ ID NO: 7 - protein, 126 aa, Arabidopsis lyrata
MASMQLRKCCNHPYLFQGAEPGPPYTTGDHLITNAGKMVLLDKLLPKLKERDSRVLIFSQMTRLLDILEDYLMYRGYLYCRIDGNTGGDERDASIEAYNKPGSEKFVFLLSTRAGGLGINLATADV

SEQ ID NO: 8 - protein, 215 aa, Prunus persica
  DISULFID (29)..(44)
  BINDING (59)..(64)
  BINDING (106)..(108)
  BINDING (113)..(113)
  BINDING (150)..(152)
MPQATMIFPILFTFFLLLSSSNAAVQDFCVADLAAPEGPAGFSCKKPASVKVNDFVFSGLGIAGNTSNIIKAAVTPAFVAQFPGVNGLGISIARLDLAVGGVVPFHTHPGASEVLIVAQGTICAGFVASDNTPYLQTLEKGDIMVFPQGLLLHFQVNGGEAPALAFASFGSASPGLQILDFALFKNDLPTEVIAQTTFLDAAQIKKLKGVLGGTN

(19) United States
(12) Patent Application Publication
(10) Pub. No.: US 2016/0108414 A1
(43) Pub. Date: Apr. 21, 2016

(54) GROWTH-RELATED ENOX PROTEINS FROM PLANTS WITH YIELD ENHANCEMENT POTENTIAL, SEQUENCES AND METHODS

(71) Applicant: Mor-NuCo Enterprises, Inc., West Lafayette, IN (US)

(72) Inventors: D. James Morré, West Lafayette, IN (US); Dorothy M. Morré, West Lafayette, IN (US)

(21) Appl. No.: 14/979,764

(22) Filed: Dec. 28, 2015

Related U.S. Application Data
(63) Continuation of application No. PCT/US2013/047569, filed on Jun. 25, 2013.

(51) Int. Cl. C12N 15/82

(52) U.S. Cl. CPC C12N 15/8238; C12N 15/827

(57) ABSTRACT

Described are compositions of matter and methods useful for increasing the yield of transgenic agricultural crops. Sequence information of ENOX proteins and methods for transfection are disclosed. Additionally, small molecule activators of ENOX proteins are disclosed. Sequence information from ENOX proteins from the yeast Saccharomyces cerevisiae, Aribidopsis thaliana and Prunus persicaria are disclosed. Transgenic microorganisms and/or plants are disclosed which may express one or more of the follow characteristics including, but not limited to, accelerated maturity, increased cell size, increased standability, increased root and xylem development, and increased yield.

### REFERENCE TO RELATED APPLICATIONS

This application is a continuation of, and claims the benefit of priority to, International PCT Application Number PCT/US2013/047569, filed on Jun. 25, 2013 which is hereby incorporated by reference in its entirety.

### FIELD OF THE INVENTION

In certain aspects, the present invention relates to the fields of genetic engineering, molecular biology, plant biology, bacteriology, and agriculture.

### BACKGROUND

Farmers may suffer low crop yields or crop failure due to many factors such as weather, insect or other animal infestation. When this occurs, it may have an economic impact on the farmer. Sometimes, a farmer may wish to re-plant a crop to mitigate his losses due to such an occurrence. At other times, a farmer may simply wish to increase his productivity by planting a second crop.

This practice of planting one or more crops during a single season is called “double-cropping” or “multiple-cropping.” Multiple-cropping allows a farmer to increase his productivity while using the same quantity of land in a given season.

However, depending on timing, there may not be enough time remaining in the season for a second crop to mature. Therefore, careful management of the planting date and harvest date of the crops is required for a successful multi-crop season.

The second crop planted may be the same as the first crop planted, or it may be different. For example, a second crop of soybeans may be planted after a first crop of soybeans, or a first crop of wheat.

In light of this background, need exists for improved and/or alternative agricultural products with increased yield and/or decreased time to maturation. Aspects of the present invention are addressed to these needs.

### SUMMARY

The present invention, in certain embodiments, describes the cloning, expression and characterization of a plant candidate constitutive ENOX (CNOX or ENOX1) protein from Arabidopsis lyrata. The gene encoding the 335 (165) amino acid protein is found in accession XP-002882467. Functional motifs characteristic of ENOX proteins previously identified by site-directed mutagenesis and present in the candidate ENOX1 protein from plants include adenine nucleotide and copper binding motifs along with essential cysteines. However, the drug binding motif (EEMTE) sequence of human ENOX2 is absent. The activities of the recombinant protein expressed in E. coli were unaffected by capsaicin, EGCg and other ENOX2-inhibiting substances. Periodic oxidative activity was exhibited both with NAD(P)H and reduced coenzyme Q as substrate. Bound copper was necessary for activity and activity was inhibited by the ENOX1-specific inhibitor simalikalactone D. Addition of melatonin phased the 24 min period such that the next complete period began 24 min after the melatonin addition as appears to be characteristic of ENOX1 activities in general. Periodic protein disulfide-thiol interchange activity also was demonstrated along with the 2 oxidative plus 3 interchange activity pattern characteristic of the 24 min ENOX1 protein period. Concentrated solutions of the purified plant ENOX1 protein formed insoluble aggregates, devoid of enzymatic activity, resembling amyloid. Activity was restored to aggregated preparations by isoelectric focusing. The above characteristics parallel those of the mammalian ENOX1 making the ENOX1 from Arabidopsis an ideal candidate to overexpress in plants as a means to increase biomass and yields.

In certain aspects, the present invention involves the cloning, transfection, and expression of ENOX proteins in hybrid organisms, such as, but not limited to bacteria, plants, and plant seeds.

Additionally, embodiments of the present invention describe a method of increasing yield in a plant by applying a small molecular weight activator of ENOX1 to the plant. The activator designated TR-III, preferably cysteine, is applied in an amount ranging from about 0.005 to 1.0 pound per acre (lb/A) as a foliar spray. Preferably, 0.01 lb/A of the cysteine is applied. In addition, the present invention provides a method of enhancing growth in plants which comprises applying cysteine as a seed treatment to a plant seed. The cysteine is applied to the seeds in an amount ranging from about 0.001 to 1 mg per g of a suitable carrier (mg/g) such as talc. Preferably, cysteine is applied between the range of 0.002 to 0.02 mg/g of talc. The present invention also provides a method of enhancing both root growth and stem diameter (increased standability) in plants which comprises applying cysteine to the plant. The cysteine is applied in an amount ranging from about 0.005 to 1.0 lb/A. Preferably, 0.01 lb/A of the cysteine is applied to the plant.

In another embodiment, the present invention discloses the cloning, expression and characterization of a plant candidate constitutive ENOX protein activated by both natural (IAA) and synthetic (2,4-dichorophenoxyacetic acid, 2,4-D) auxin plant growth regulators with an optimum of about 1 μM in certain embodiments, and higher concentration being less effective. Functional motifs characteristic of the ENOX1 proteins of plants previously identified by site-directed mutagenesis and present in the candidate auxin-activated ENOX (dNOX) include adenine nucleotide and copper binding motifs along with essential cysteines in addition to a previously identified auxin binding motif. Periodic oxidative activity was exhibited by both the oxidative [NAD(P)H and reduced coenzyme Q as substrate] as well as for protein disulfide interchange to yield the 2 oxidative plus 3 interchange activity pattern characteristic of the 24 min periodicity of other growth-related ENOX proteins. Bound copper was necessary for activity and activity was inhibited by the ENOX1-specific inhibitor simalikalactone D. Preparations were devoid of activity in the absence of auxin. The inactive auxin 2,3-D was without effect as were ENOX2 inhibitors. Concentrated solutions of the purified plant ENOX1 protein formed insoluble aggregates, devoid of enzymatic activity, resembling amyloid. Activity was restored to aggregated preparations by isoelectric focusing. The above characteristics which parallel those of the mammalian ENOX1 make the plant dNOX a second candidate to overexpress in plants as a means to increase biomass and yield.

Additional summaries are provided in the claims appended hereto, each of which is to be considered a summary of an embodiment of the present invention.

The foregoing and still further aspects of the invention will become more apparent from the following detailed description and accompanying figures.

### BRIEF DESCRIPTION OF THE DRAWINGS

FIG. 1. Cell growth correlates with ENOX1 activity.

FIG. 2. Human ENOX1 overexpression increases cell size.

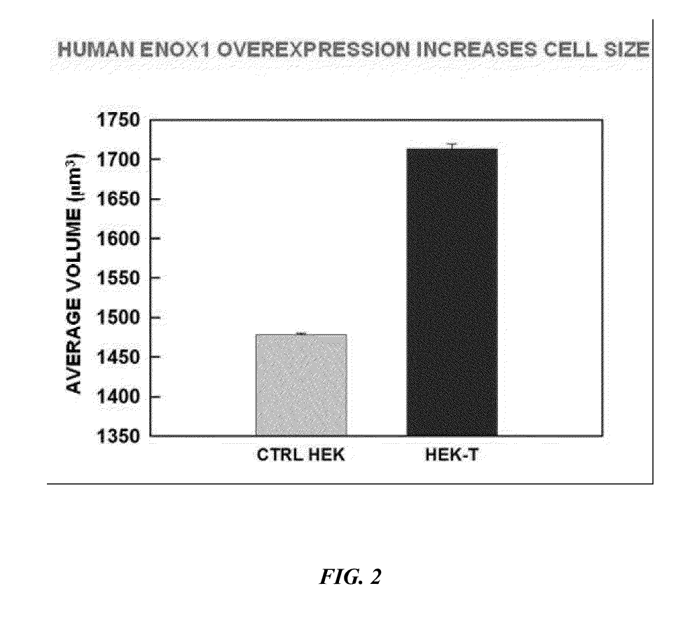

FIG. 3. NCBI Reference Sequence: XP_002882467.1 (SEQ ID NO 5). Alignment of Saccharomyces cerevisiae YML117w (SEQ ID NO: 6) and Arabidopsis ENOX1 (SEQ ID NO: 7). There is 37% ( 16/43) identity and 58% ( 25/43) similarity between recombinant Arabidopsis ENOX1 amino acids 84 to 126 and YML117W amino acids 932-968.

FIG. 4. Expression of 14 kD recombinant Arabidopsis ENOX1 shown on 15% SDS-PAGE with silver staining. Lanes 1 and 2: Whole cells of pET11a-AraENOX1 transformed E. coli (2 μl); lane 3: Pellet of French pressed pET11a-AraENOX1 transformed E. coli (2 μl). The expressed recombinant Arabidopsis ENOX1(arrow) was found in the pellet of French pressed E. coli.

FIG. 5. Continuous trace showing the decrease in A₃₄₀ as a measure of consumption of NADH over 12 min for a fraction of IEF purified Arabidopsis ENOX1. The assay conditions were as described (Jiang, Z., Gorenstein, N. M., Morré, D. M. and Morré, D. J. 2008. Biochemistry 47:14028-14038) except that the NADH concentration was 0.75 mM and the data were collected automatically and stored using a SPECTRA max 340PC microplate reader. The mixture contained ca. 20 μg ENOX1 in a total volume of 200 μl.

FIG. 6. NADH oxidase activity of IEF-purified recombinant ENOX1 of Arabidopsis. Illustrated is the oscillatory pattern of 5 maxima. The major maxima separated by 6 min are indicated by maximum labeled 1 and 2. The three minor maxima that follow are separated from the major maxima and each other by 4.5 min creating the 24 min period [6+(4.5×4)=24].

FIG. 7. The NADH oxidase activity of IEF-purified recombinant ENOX1 of Arabidopsis and response to 1 μM melatonin. After addition of melatonin, new maxima appear 24 min following melatonin addition (arrow), an ENOX1 characteristic.

FIG. 8. Protein disulfide-thiol interchange activity of IEF-purified recombinant Arabidopsis ENOX1 measured from the cleavage of a dithiodipyridine (DTDP) substrate. An oscillatory activity was observed with the activities most strongly associated with the three maxima separated by 4.5 min rather than with the two maxima separated by 6 min.

FIG. 9. Ability of recombinant Arabidopsis ENOX1 to oxidize hydroquinone (reduced coenzyme Q) measured either by an increase in A₄₁₀ (A) or a decrease in A₂₉₀ (B). As with NADH oxidation of FIG. 6, the activity oscillates with prominent maxima separated by 6 min (arrows) to create a 24 min period containing 3 additional maxima separated by 4.5 min (total of 5 maxima).

FIG. 10. Purification and activation of recombinant Arabidopsis ENOX1 by isoelectric focusing.

FIG. 11. Inhibition of recombinant Arabidopsis ENOX1 by the specific ENOX1 quassinoid inhibitor simalikalactone D.

FIG. 12. NADH oxidase activity of recombinant Arabidopsis ENOX1 diminished with TFA+bathocuproine. A. In the presence of TFA, the 24 min period was unaffected. B. When assayed with TFA and bathocuproine, the 24 min period was much reduced. C. Removal of bathocuproine by dialysis and re-addition of copper restored full activity.

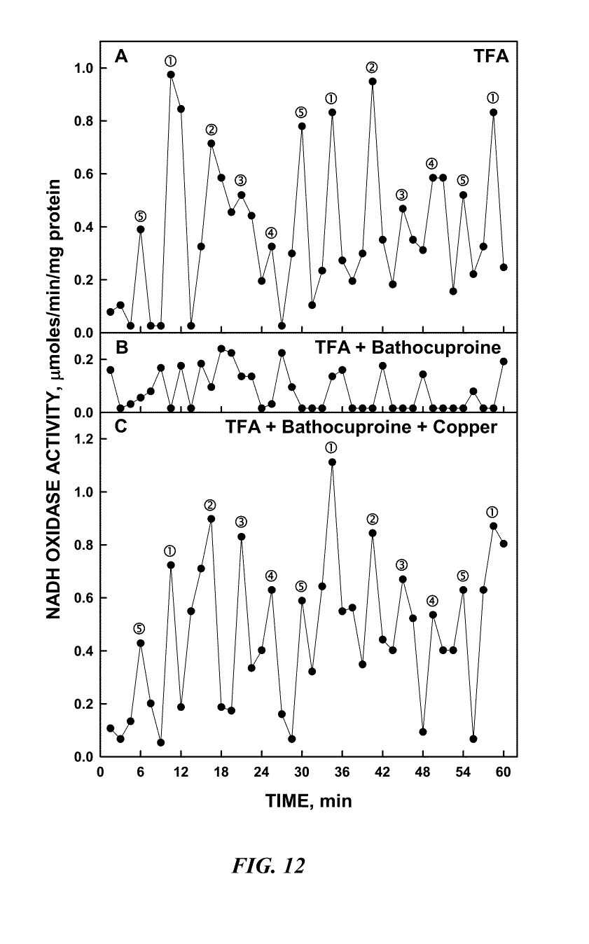

FIG. 13. NADH oxidase activity of Arabidopsis ENOX1 when assayed in D₂O exhibited an increase in period length from 24 min to 30 min. The effect of heavy water to increase period length is one of the hallmarks of the biological clock.

FIG. 14. Stimulation of NADH oxidation by cysteine is specific for maximum \textcircled{3} of the ENOX1 activity cycle of recombinant ENOX1 protein expressed in bacteria.

FIG. 15. Soybean seeds were germinated in vermiculite in darkness and 2 cm hypocotyl sections were harvested just below the hook. These were homogenized, plasma membranes were prepared, and ENOX1 activity was assayed.

FIG. 16. As in FIG. 15, except leaf tissue (1^{st} and 2^{nd} trifoliates) of soybean plants grown in the greenhouse after 1 month.

FIG. 17. Soybean plant seeds were geminated in vermiculite in darkness and after 7 days, seedlings were excised above the roots and placed in water contained in vials in the light.

FIG. 18. As in FIG. 17, except untreated seeds were germinated and excised shoots were transferred to TR-III solutions of different concentrations prepared in water.

FIG. 19. Plants were grown from treated seeds in the greenhouse.

FIG. 20. As in FIG. 19, except plants were from untreated seed and sprayed with different rates of TR-III. The experiment is still in progress but epicotyl enlargement was observed at 0.01 lb/A TR-III as in the past with little or no effect from 0.001 or 0.1 lb/A.

FIG. 21. Pods per plant of soybeans in a field experiment comparing no TR-III (solid symbols) with 0.01 lb/A TR-III (open symbols, dashed lines) as a foliar spray applied July 3.

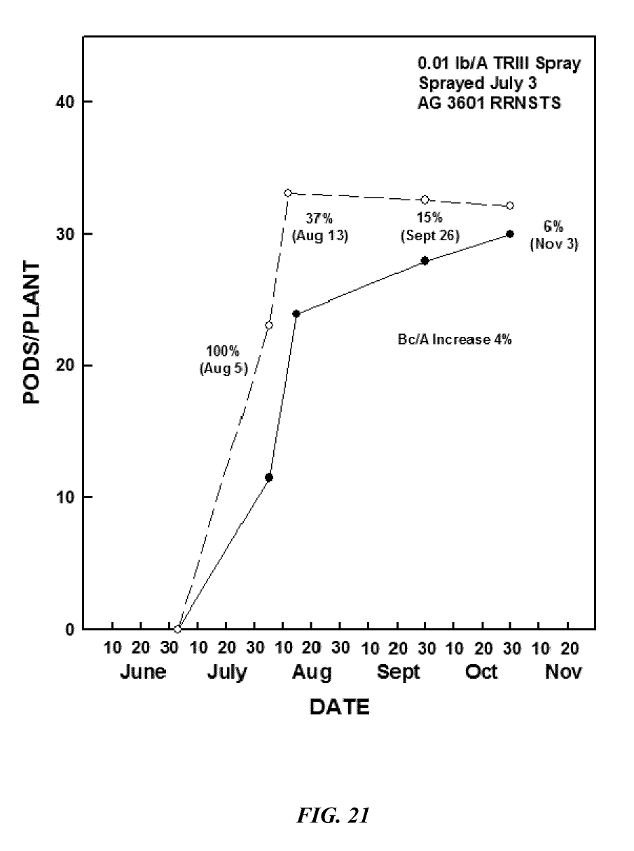

FIG. 22. Increase in secondary xylem of soybean stem of plants grown from seeds treated with talc comparing no TR-III, TR-III 1:50 and TR-III 1:500.

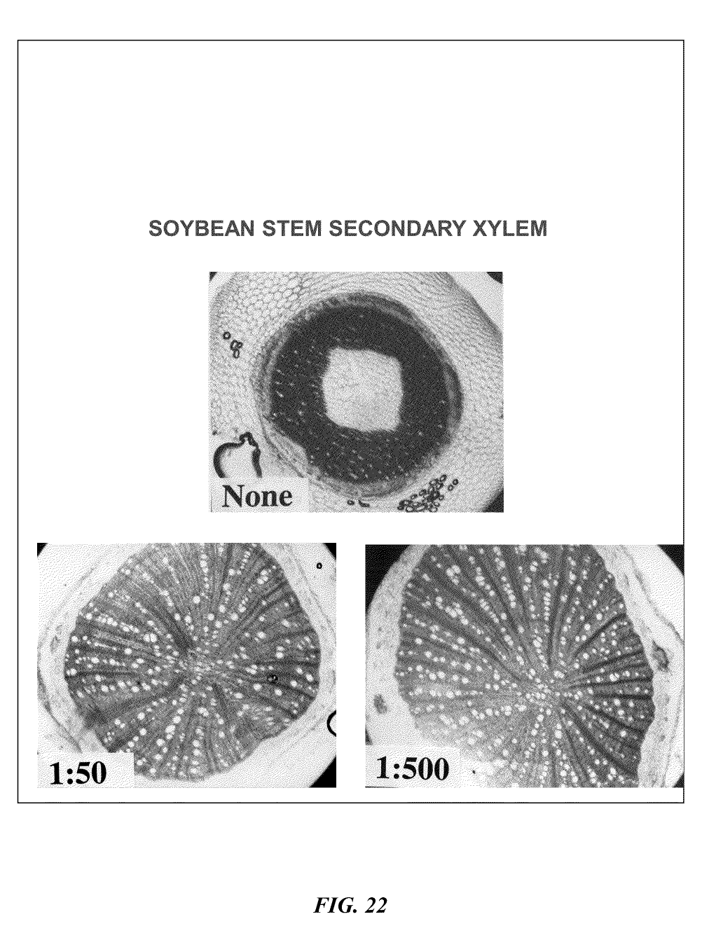

FIG. 23. Standability of soybeans from the field experiment of FIG. 21. No TR-III plants (left) were severely lodged. TR-III-treated plants (right) did not lodge.

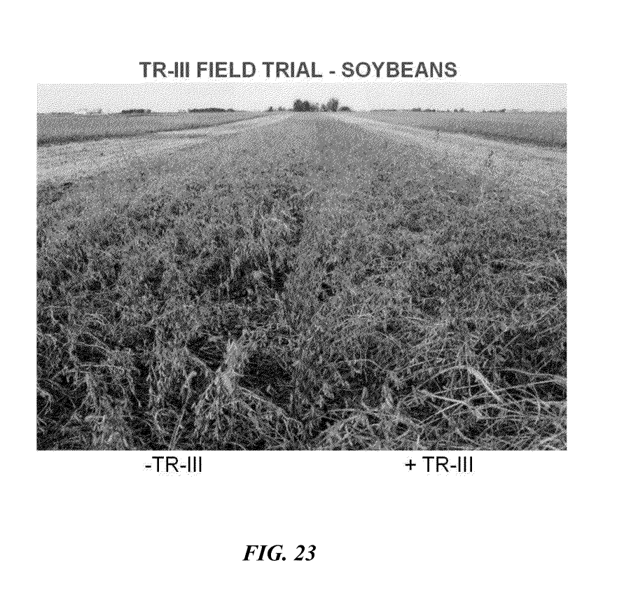

FIG. 24. Sequence of the recombinant auxin-activated ENOX protein (ABP-20) (SEQ ID NO: 8).

FIG. 25. Expression of 20 kD recombinant ABP-20 shown on 15% SDS-PAGE with silver staining. Lane 1: Whole cells carrying vector pET-11b; lane 2: Whole cells of pET11b-ABP-20 transformed E. coli (2 μl); lane 3: Supernatant of French pressed pET11b-ABP-20 transformed E. coli (2 μl): lane 4: Pellet of French pressed pET11b-ABP-20 transformed E. coli (2 μl). The expressed recombinant ABP-20 was found in the pellet of French pressed E. coli (arrow).

FIG. 26. NADH oxidase activity of IEF purified recombinant ABP-20. 2,4-dichlorophenoxyacetic acid (2,4-D) (1 μM) was added at 60 min to activate the enzyme. Illustrated is the oscillatory pattern of 5 maxima. The major maxima separated by 6 min are indicated by single arrows. The three minor maxima that follow are separated from the major maxima and each other by 4.5 min creating the 24 min period [6+(4.5×4)]=24].

FIG. 27. As in FIG. 26, except activation by 10 μM indole-3-acetic acid added after 60 min (arrow).

FIG. 28. Protein disulfide-thiol interchange activity of IEF-purified recombinant ABP-20 measured from the cleavage of a dithiodipyridine (DTDP) substrate. 2,4-D (1 μM) was added at 60 min to activate the enzyme. An oscillatory activity was observed with the activities were most strongly associated with the three maxima separated \textcircled{3}, \textcircled{4} and \textcircled{5} by 4.5 min rather than with the two maxima \textcircled{1} and \textcircled{2} separated by 6 min.

FIG. 29. Ability of recombinant ABP-20 to oxidize hydroquinone (reduced coenzyme Q=ubiquinol) measured either by an increase in A₄₁₀ (A) or a decrease in A₂₉₀ (B). As with NADH oxidation of FIG. 6, the activity oscillates with prominent maxima separated by 6 min (arrows) to create a 24 min period containing 3 additional maxima separated by 4.5 min (total of 5 maxima). 2,4-D (1 μM) was added at 60 min to activate the enzyme.

FIG. 30. NADH oxidase activity of recombinant ABP-20 diminished with TFA+bathocuproine. A. In the presence of TFA, the 24 min period was unaffected. B. When assayed with TFA and bathocuproine, the 24 min period was much reduced. C. Removal of bathocuproine by dialysis and re-addition of copper restored full activity. 2,4-D (1 μM) was added from the beginning to activate the enzyme.

### DETAILED DESCRIPTION

For the purposes of promoting an understanding of the principles of the invention, reference will now be made to certain embodiments and specific language will be used to describe the same. It will nevertheless be understood that no limitation of the scope of the invention is thereby intended, such alterations and further modifications, and such further applications of the principles of the invention as described herein being contemplated as would normally occur to one skilled in the art to which the invention relates.

Articles and phrases such as, “the”, “a”, “an”, “at least one”, and “a first”, “comprising”, “having”, and “including” here are not limited to mean only one, but rather are inclusive and open ended to also include, optionally, two or more of such elements and/or other elements. In terms of the meaning of words or terms or phrases herein, literal differences therein are not superfluous and have different meaning, and are not to be synonymous with words or terms or phrases in the same or other claims.

This patent is predicated on the potential utility of a family of growth-related cell surface NADH oxidases (ECTO-NOX=ENOX) proteins of plants, animals and yeasts. ENOX (ECTO-NOX=Ecto-Nicotinamide Dinucleotide Oxidase Disulfide Thiol Exchange) proteins exhibit a cyanide-insensitive, time-keeping reduced coenzyme Q (CoQH₂) (NAD(P)H) oxidase (NOX) activity and a protein disulfide-thiol interchange activity that alternate (Morré, D. J. 1998. In: Asard, H., Bérczi, H. and Canbergs, R., eds., Plasma Membrane Redox Systems and Their Role in Biological Stress and Disease, Kluwer, Dordrecht, pp. 121-156; Morré, D. J. and Morré, D. M. 2003. Free Radical Res. 37: 795-808). The ENOX proteins carry out plasma membrane electron transport and a protein disulfide-thiol interchange activity, the latter of which drives cell enlargement. We have identified and cloned the constitutive ENOX (ENOX1) proteins from Arabidopsis, yeast (Saccharomyces cerevisiae) and human as well as a cancer-specific ENOX2 form also of human origin. We have evidence that appropriate overexpression of one or more of these ENOX family members in agronomic crops would lead to substantially increased yields. The ECTO designation derives from their external location on the outer surface of the plasma membrane and to distinguish them from all other cellular NADH oxidases. This external location and alternation of oxidative and protein disulfide interchange activities has been demonstrated for a wide range of animal and plant tissues and cell lines (D. J. Morré and D. M. Morré, 2012, ECTO-NOX Proteins, Springer, New York, 507 pp). Of the ENOX proteins, the constitutive form, CNOX or ENOX1 emerges as having the greatest utility for overexpression in production agriculture.

Our interest in the ENOX1 protein is predicated on nearly 3 decades of published basic research indicative of a vital and essential role for ENOX1 to drive cell enlargement in both plant and animal cells (D. J. Morré and D. M. Morré, 2012, ENOX Proteins, Springer, New York, 507 pp). Approximately 100 peer reviewed journal papers related to the general subject of understanding the enlargement phase of cell growth have been published going back to the early 1960s and the ENOX proteins involved beginning in the mid-1970s to mid-1980s.

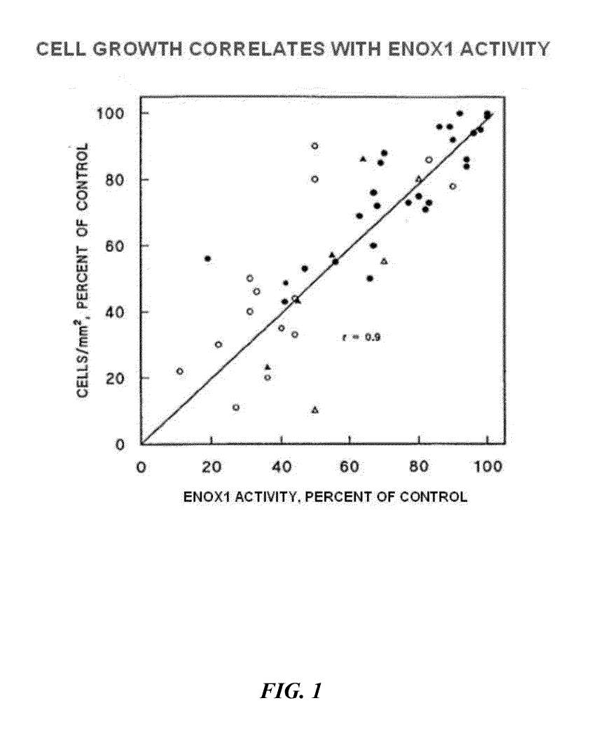

The laboratory conclusions are based primarily on three lines of evidence:
1. A strong correlation between rate of cell enlargement and ENOX1 activity (FIG. 1).
2. Inhibition of cell enlargement (and growth) by relatively specific inhibitors of both ENOX1 and cell enlargement (Morré, D. J. and Greico, P. A. 1999. Int. J. Plant Sci. 160:291-297).
3. Overexpression of cloned ENOX1 in a mammalian cell line (HEK) that resulted in increased rates of cell enlargement and increased cell volume (Bosneaga, E. and Tang, X. Unpublished).

The growth-related cell surface ENOX1 proteins that are essential to the elongation (cell expansion) phase of cell growth were first cloned in the human ENOX1 gene (Jiang, Z., Gorenstein, N. M., Morré, D. M. and Morré, D. J. 2008. Biochemistry 47:14028-14038) which was overexpressed in Williams 82 soybeans. The result was shorter internodes, an overall increase of about 2.5 pod bearing nodes per plant, an increase in plant height of about 3 inches, an increase in xylem and stem diameter and an increased yield of 15% resulting primarily from the extra pod-bearing nodes. In the meantime, the ENOX1 from the yeast Saccharomyces cerevisiae was cloned as was the ENOX1 from Arabidopsis as more likely candidates for overexpression in plants. Also cloned was a second ENOX1-like protein unique to plants where activity is dependent upon the presence of auxins either natural or synthetic (dNOX).

Additionally, we have discovered a proprietary small molecule activator of ENOX1 that is effective as a seed treatment and has given substantially increased yields at little or no extra cost especially with double crop soybeans. An advantage of the seed treatment is that it accelerates plant development with the shortening photoperiod of late summer to maximize pod production in the time available to produce a crop.

### Overexpression Mammalian ENOX1

The gene from the human genome for the constitutive ENOX1 protein was cloned by Jiang et al. (2008) designated as ENOX1 (formerly CNOX) similar to the proliferating-inducing gene 38 protein. The protein was cloned and expressed in E. coli (NCBI accession number for the protein is AB028524).

When expressed in bacteria with a NusA tag, cENOX1 had activity characteristics of ENOX1 proteins from other mammalian or plant sources. In the human genome, the gene is located on the chromosome 13 (13q 14.11) and codes an open reading of 643 amino acids. A gene coding for cENOX1 is present in genomes of all so far sequenced Vertebrata and insect species and the protein is highly conserved. In Mammalia with the XY system of sex determination, the gene has autosomal localization of the X chromosome. Despite having common functional motifs, the similarity was found between the mammalian ENOX1 and the ENOX1 in plants, yeast, or prokaryotes nor does the plant and yeast ENOX1 counterparts have sequence similarity to the human gene.

To reduce the concept to practice the mammalian ENOX1 gene was introduced into soybeans by the Gene Transfection Service of the Iowa State University, Ames, Iowa. The regulated material was released for field trial at two locations, Indiana and Illinois in both 2011 and 2012. The release site was identified using flags and stakes with allowed zones as borders. At the end of the growing season, all regulated material except for harvested seeds was left at the regulated site and destroyed by tillage.

Phenotypic Designation Name: CXOX2008

Identifying Line(s): ICIA0001, ICIA002, ICIA003

Construct(s): Agrobacterium tumefaciens, disarmed

Phenotype Description:

| | |
|---|---|
| A description of the anticipated | Cells elongate faster and stem length is |
| or actual expression of the altered | increased to where the plant reaches |
| genetic material in the regulated | maturity sooner as a result of earlier |
| article and how that expression | flowering. Additionally, yield and |
| differs from the expression in | standability are enhanced. |
| the non-modified parental organism. | |

### Genotype(s):

### Gene(s) of Interest:

Promoter: 35S from Cauliflower mosaic caulimovirus—Enhanced 35S

Enhancer: TEV from Tobacco etch polyvirus—Additional upstream sequence from 35S promoter

Gene: CNOX from Homo sapiens—gene designed using the Condon usage table

Terminator: NOX from Agrobacterium tumefaciens—NOX 3′ from T-DNA

### Selectable Marker:

Promoter: 35S from Cauliflower mosaic caulimovirus—Enhanced 35S

Enhancer: TEV from Tobacco etch polyvirus—Additional upstream sequence from 35S promoter

Gene: herbicide resistance from Streptomyces hygroscopicus—selectable marker

Terminator: NOX from Agrobacterium tumefaciens—NOX 3′ from T-DNA

### Performance Evaluations of the 2011 Field Trials of the CNOX (ENOX1) Synthetic Gene Construct Expressed in Williams 82

Approximately one-half of the regulated material available for evaluation in 2011 was distributed between the two release sites, approximately two-thirds for the Atlanta, Ind. site and approximately one-third for a Downs, Ill. site.

All transgenic plants were collected and harvested by hand and compared to the Iowa State University Williams 82 variety plus comparable numbers of Williams 82 plants from Indiana and Missouri seed stocks (Table 1). Phenotypic parameters evaluated are listed in Tables 2 and 3. Comparisons were with Williams 82 plants grown from seed obtained from all four sources (Table 1). Fifty-five (55) Williams 82, wild type non-transgenic plants, divided equally among the four sources and grown under conditions identical to the transgenic plants were harvested from the Atlanta, Ind. release site and twenty-four (24) Williams 82 plants were harvested from the Downs, Ill. site. No differences were noted among the four sources of Williams 82 plants. Aggregate data are presented as means±standard deviations among the different Williams 82 sources.

One hundred fifteen (115) transgenic plants from 18 different events were harvested and analyzed from the Atlanta, Ind. site and eleven (11) plants from 5 events were harvested from the Downs, Ill. site. Not all events produced plants. All transgenic plants reaching maturity were harvested and included in the final data summary. Findings given in Tables 2 and 3 are averages of all events producing plants ±standard deviations among events.

Plant height was largely unaffected comparing wild type Williams 82 and transgenic (Table 2). Results from the Atlanta location (Table 2A) revealed an 11% increase in pod-bearing nodes, a 20% increase in filled pods/pod-bearing node and a small, marginally significant, increase in weight per bean. These three parameters (increase in pod-bearing nodes, increase in filled pods/nod and increased weight per bean) provided a combined increase of 33% that compared favorably with the increase in total weight of beans per plant of 32%.

Similar results were observed with the material collected from the Downs, Ill. site (Table 2B).

Other parameters comparing the transgenic plants with Williams 82 plants (Table 3) were largely unchanged. Degree of branching, beans/pod, empty pods/plant (as percent of total pods; empty pods were excluded from the filled pod count) were not different either with plants from the Atlanta, Ind. release site (Table 3A) or from the Downs, Ill. release site (Table 3B). Only with stem diameter measured at the 9^{th} internode from the top of the plant, approximately midway from the top to the base, were differences noted. The stems of the transgenic plants were, on average, 15% thicker (stem diameter was increased by 15%) with transgenic plants from the Atlanta site and 7% thicker with transgenic plants from the Downs, Ill. site, compared to Williams 82 plants from the same locations.

With the four Williams 82 plantings at the Atlanta release site and three of the transgenic plantings at the Atlanta release site contained 23 or more (31±8) contiguous plants. Estimates from these plants revealed a calculated yield of 58 bu/A for Williams 82 and 83 bu/A for the transgenics with an overall increase of 43% (Table 2A).

The absolute calculated yields are based on an average plant spacing of 5.5 inches apart (4 inches apart with a germination of 73%) and a row spacing of 30 inches. The Williams 82 lots and transgenic event plots included in the comparison had nearly identical plant spacings and also were in 30 inch rows. There were insufficient contiguous plants at the Downs, Ill. release site to permit similar meaningful calculations of yield per acre.

The two principal parameters contributing to increased yield (increased numbers of pod-bearing nodes with correspondingly shorter internodes and increased numbers of pods per node) were very reproducible among the four Williams 82 sources and among all events with small standard deviations and high statistical significance for both release sites. By comparing isolated plants from both Williams 82 and the transgenics, the principal parameters contributing to increase yield were unaffected by plant spacing within the row. Contributory factors to the apparent 30 to 40% increase in yield other than the transgene cannot be ruled out, however.

TABLE 1
Plants Harvested and Analyzed.

```
A. Atlanta, IN
      Williams 82 Non-transgenic
            55 Plants from 4 seed sources:
                  Iowa State
                  Iowa State Greenhouse 2010
                  Indiana
                  Missouri
      Transgenic: 115 plants from 18 events
B. Downs, IL
      Williams 82 Non-transgenic
            24 Plants from 4 seed sources (above)
      Transgenic: 11 Plants from 5 events
```

TABLE 2
Summary of Harvest Data.

| | | | | | |
|---|---|---|---|---|---|
| | | Filled | | | |
| | | Pods/Pod | | | |
| Plant Height | Pod Bearing | Bearing | Wt/100 Beans | Total Beans | |
| (In) | Nodes | Nodes | (g) | (g/plant) | Bu/A |

A. Atlanta, IN

| | | | | | | |
|---|---|---|---|---|---|---|
| Williams 82 | 34.3 ± 1.2 | 15.3 ± 0.9 | 2.0 ± 0.1 | 15.43 ± 0.49 | 51.85 ± 10.1 | 58 ± 16 |
| Transgenic | 35.6 ± 2.1 | 17.2 ± 1.1 | 2.4 ± 0.2 | 15.8 ± 0.57 | 68.7 ± 19.4 | 83 ± 14 |
| | 4% | 11% | 20% | 3% | 32% | 43% |
| | | p = 0.001 | p = 0.001 | p = 0.09 | p = 0.017 | p 0.01 |

B. Downs, IL

| | | | | | |
|---|---|---|---|---|---|
| Williams 82 | 35.6 ± 2.0 | 17.1 ± 1.1 | 2.4 ± 0.15 | 17.1 ± 0.56 | 96.5 ± 23.0 |
| Transgenic | 35.3 ± 1.4 | 19.2 ± 1.5 | 2.9 ± 0.3 | 18.37 ± 0.68 | 133.4 ± 25.9 |
| | 0% | 12% | 21% | 7% | 38% |
| | | p = 0.035 | p = 0.01 | p = 0.01 | p = 0.035 |

TABLE 3
Summary of Harvest Data.

| | | | | Stem Diameter |
|---|---|---|---|---|
| | | | Empty | (9 internodes |
| Branches | Branches | | Pods/Pant | from top) |
| <6″ | >6″ | Beans/Pod | (%) | (cm) |

A. Atlanta, IN

| | | | | | |
|---|---|---|---|---|---|
| Williams 82 | 4.8 ± 1.2 | 7.6 ± 2.9 | 2.36 ± 0.07 | 4.0 ± 1.2 | 0.61 ± 0.02 |
| Transgenic | 4.4 ± 1.9 | 8.4 ± 3.3 | 2.39 ± 0.14 | 3.7 ± 1.5 | 0.70 ± 0.025 |
| | | | | | 15% |
| | | | | | p = 0.001 |

B. Downs, IL

| | | | | | |
|---|---|---|---|---|---|
| Williams 82 | 6.4 ± 2.4 | 9.2 ± 1.3 | 2.31 ± 0.13 | 1.3 ± 0.7 | 0.76 ± 0.005 |
| Transgenic | 8.5 ± 3.1 | 7.2 ± 1.0 | 2.35 ± 0.16 | 2.1 ± 1.1 | 0.81 ± 0.05 |
| | | | | | 7% |
| | | | | | p = 0.0564 |

### Performance Evaluations of 2012 Field Trials of the Human ENOX1 Synthetic Gene Construct Expressed in Williams 82

In contrast to 2011, the transgenic plants were taller although individual heights overlapped with Williams 82 (the tallest plants were 30 inches in both but the Williams 82 contained more shorter plants (Table 4)). Node length was not increased. As a result, nodes/plant were increased, a feature consistent with 2012. Also increased was pod-bearing nodes/plant. Pods/node, empty pods, pods/pod bearing node, nodes without pods, and stem diameters were unchanged.

Pods per plant compared to the Williams 82 average were increased by 16% and total weight of soybeans by 15%. This agrees with the 15% increase from Atlanta of 60 bu/A for ST104-2-4 GH2010 (Row 11) compared to 52±4 bu/A for the average of Williams ISU GH2010 (Row 1+Row 2) and Williams ISU GH2010 (Row 2B1) with Williams ISU GH2010 (Row 1+Row 2) yielding closer to ST 104-2-4 GH2010 Row 11 than Williams 82 GH2010 (Row 2B1) in parallel in both locations.

TABLE 4
Summary of Findings Transgenic Soybeans from El Paso, Illinois, harvested 2012

| | | Williams 82 | |
|---|---|---|---|
| | Transgenic | Iowa State | Williams 82 |
| | ST 104-2-24 | University | Iowa State University |
| | GH2010 Row 11 | GH2010 Row 2B1 | GH2010 Row 1 + Row 2 |
| Height (in) | 26 ± 3* | 23 ± 3 | 22 ± 5 |
| Pods (Total) | 824* | 697 | 727 |
| Pods/Plant | 27.5 | 23.2 | 24.2 (23.7 ± 0.5) |
| Nodes (Total) | 465 | 397 | 408 |
| Nodes/Plant | 15.5* | 13.2 | 13.6 |
| Internode Length (in) | 1.67 | 1.74 | 1.58 |
| Pods/Node | 1.8 | 1.8 | 2.0 |
| Pod Bearing Nodes | 326* | 265 | 287 |
| Pod Bearing Nodes/Plant | 10.9* | 8.8 | 9.6 |
| Nodes/Plant without Pods | 4.6 | 4.7 | 4.0 |
| Pods/Pod Bearing Node | 2.5 | 2.8 | 2.9 |
| Empty Pods | 5 | 20 | 14 |
| Branches | 17 | 15 | 33 |
| Stem Diameter (cm) | | | |
| Below first node | 0.8 | 0.71 | 0.86 |
| Between Nodes 7 and 8 | 0.66 | 0.61 | 0.68 |
| Seed Weight (Total) (g) | 235 | 189 | 220 |
| Seed Weight per Plant (g) | 7.8* | 6.3 | 7.3 (6.8 ± 0.5) |

*Significant differences

Search for Candidate Constitutive ENOX1 (ENOX1) from Plants.

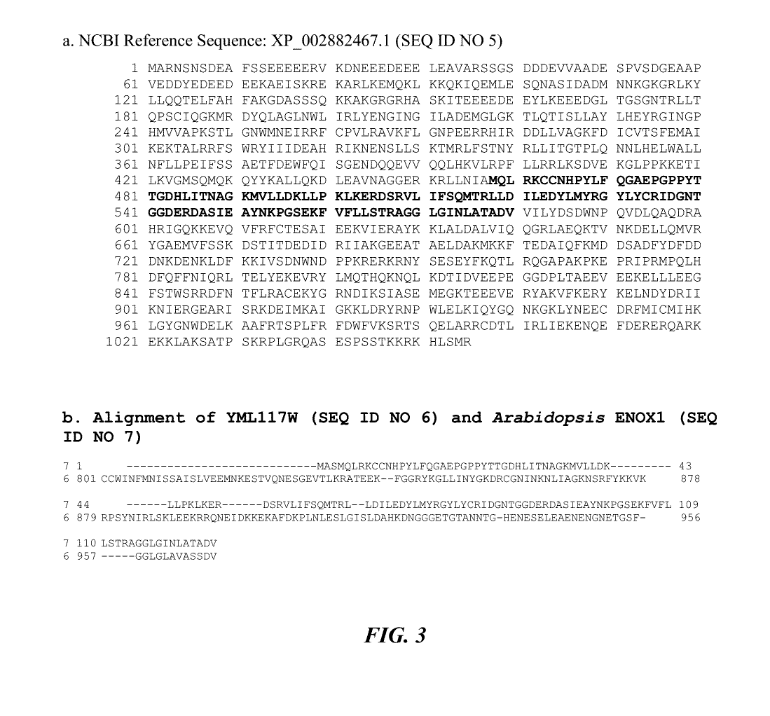

Protein BLAST (Basic Local Alignment Search Tool) with either ENOX1 or ENOX2 sequences as a query was used for similarity searches in different databases (non-redundant protein sequences, UniProt, EST and others) (Altschul, S., Madden, T. L., Schïffer, A. A., Zhang, J., Zhang, Z., Miller, W. and Lipman, D. J. 1997. Nucleic Acids Res. 25:3389-3402) with no plant proteins having significant similarity being found. However, sequence of a cloned ENOX1 from Saccharomyces cerevisiae (FIG. 3) did reveal significant homology.

The homologous protein from Arabidopsis lyrata was selected for evaluation as a candidate for the constitutive ENOX1 from plants.

Plasmids Construction:

Plasmids carrying the Arabidopsis ENOX1 (M458 to V580 of hypothetical protein ARALYDRAFT_477943 [Arabidopsis lyrata aubsp. Lyrata] XP_002882467) sequence were prepared by inserting the pET11a vector (between NheI and BamHI sites) with the Arabidopsis ENOX1 sequence. The Arabidopsis ENOX1 sequence was synthesized by GenScript USA Inc. (Piscataway, N.J.). DNA sequences of the ligation products (pET11a-AraENOX1) were confirmed by DNA sequencing.

Expression of Recombinant Arabidopsis ENOX1:

The pET11a-AraENOX1 was transformed to BL21 (DE3) competent cells. A single colony was picked and inoculated into the 5 ml LB+ampicillin (LB/AMP) medium. The overnight culture (1 ml) was diluted into 100 ml LB/AMP media (1:100 dilution). The cells were grown with vigorous shaking (250 rpm) at 37° C. to an OD₆₀₀ of 0.4-0.6 and IPTG (0.5 mM) was added for induction. Cultures were collected after 5 h incubation with shaking (250 rpm) at 37° C.

Cells were centrifuged at 5,000 g for 6 min. Pellets were then resuspended in 20 mM Tris-HCl, pH 8.0, containing 0.5 mM PMSF, 1 mM benzamidine and 1 mM 6-aminocaproic and lysed by three passages through a French pressure cell (SLM Aminco) at 20,000 psi. Expression of the recombinant Arabidopsis ENOX1 of about 14 kDa was confirmed by SDS-PAGE with silver staining. Transformed cells were stored at −80° C. in a standard glycerol stock solution. The Arabidopsis ENOX1 proteins were further purified on Criterion IEF gels (Bio-Rad, Hercules, Calif.). The IEF gel was cut into seven equal segments. The pH represented by each slice was based on IEF standards (Bio-Rad). The slices were soaked in 15 mM Tris-Mes buffer, pH 7, at 4° C. for overnight with shaking. The gel-free extracts were assayed for ENOX1 activity.

Protein Determination.

Protein concentrations were determined by the bicinchoninic acid (BCA) method (Smith, P. K., Krohn, R. I., Hermanson, G. T., Mailia, A. K., Gartner, F. F., Provenzano, M. D., Fujimoto, E. K., Groeke, N. M., Olson, B. J. and Klenk, D. C. 1985. Anal. Biochem. 150: 70-76) (BCA Protein Asay Kit, Thermo Scientific, Rockford, Ill., USA) with bovine serum albumin as the standard.

Enzyme Activity Assays.

Oxidation of NADH was determined spectrophotometrically from the disappearance of NADH measured at 340 nm in a reaction mixture containing 25 mM Tris-MES (pH 7.2), 100 μM GSH, 1 mM KCN to inhibit mitochondrial oxidase activity, 150 μM NADH and the enzyme at 37° C. with temperature control (±0.5° C.) and stirring. Prior to assay, 1 μM reduced glutathione was added to reduce the protein in the presence of substrate. After 10 min, 0.03% hydrogen peroxide was added to reoxidize the protein under renaturing conditions and in the presence of substrate to start the reaction. Activities were measured using paired Hitachi U3210 or paired SLM Aminco 2000 spectrophotometers both with continuous recording. Assays were run for 1 min and were repeated on the same sample at intervals of 1.5 min for the times indicated. An extinction coefficient of 6.22 cm⁻¹ mM⁻¹ was used to determine specific activity.

Oxidation of reduced coenzyme Q₁₀ (CoQ₁₀H₂) was measured as the disappearance of CoQ₁₀H₂ at both 290 nM and 410 nM (19). The reaction was started with the addition of 40 μl of 5 mM Q₁₀H₂(Tischcon Corp., Westbury, N.Y.). An extinction coefficient of 0.805 mM⁻¹ cm⁻¹ was used to calculate the rate of Q₁₀H₂ oxidation.

Protein disulfide-thiol interchange was determined spectrophotometrically from the increase in absorbance at 340 nm resulting from the cleavage of dithiodipyridine (DTDP (Morré, D. J., Gomez-Rey, M. L., Schramke, C., Em, O., Lawler, J., Hobeck, J. and Morré, D. M. 1999. Mol. Cell. Bochem. 200: 7-13). DTDP cleavage was buffered (50 mM Tris-MES, pH 7). The assay was preincubated (1 h at room temperature) with 0.5 μmoles 2,2′-dithiodipyridine (DTDP) in 5 μl of DMSO to react with endogenous reductants present with the plasma membranes. After 10 min, a further 3.5 moles DTDP were added in 35 μl DMSO to start the reaction. The final reaction volume was 2.5 nil. The reaction was monitored from the increase in absorbance at 340 nm. Specific activities were calculated using a milimolar absorption coefficient of 6.21.

Removal of Copper (II) from ENOX1.

IEF purified ENOX1 was concentrated to 0.7 mg/ml by using a Centricon concentrator (Millipore Corporation, Danvers, Mass.) fitted with a 10,000 nominal molecular weight limit ultracel YM membrane. Samples (50 μl) were combined with 1 μl of trifluoroacetic acid (TFA) in the presence or absence of 15 μl 10 mM bathocuproine. After 2 h of incubation at room temperature, the samples were dialyzed (Spectra/Pro Dialysis membrane, molecular weight cut-off 6-8,000, Spectrum Laboratories (Rancho Dominguez, Calif.) against 20 mM Tris-HCl, pH 8, at 4° C. overnight.

Activation of ENOX1 by Cysteine (TR-III).

To activate plant ENOX1 using cysteine (TR-III), the cysteine is applied directly to the plant as solution or powder, or in other suitable forms. The cysteine is preferably applied in an amount from between about 0.005 to 1.0 lb/a. In the preferred embodiment, 0.01 lb/A of cysteine is applied. In addition, the cysteine may be applied as a spray, both alone or in combination with other materials such as a herbicide.

In addition to applying the cysteine to the plant, the present invention provides for applying cysteine as a seed treatment to a plant seed before planting to enhance growth. The application of cysteine to a seed produces yield increases in row crops such as soybeans. The cysteine is preferably applied to the seed in an amount from about 0.001 to 1 mg per g of a suitable carrier. For example, one suitable carrier is talc. Specifically, the cysteine is applied in an amount from about 0.002 to 0.02 mg per g of talc. The cysteine may be applied to the seeds as a spray, dust, oil or in any other suitable form or method of application. The cysteine may also be applied in combination with a fungicide, insecticide or fertilizer. The cysteine may also be applied as a seed coating in a powder, dust, slurry, or liquid form. In one embodiment the cysteine is applied to the seed in combination with other compounds such as with a fungicide, with an insecticide or with a fertilizer. Preferably, the plant seed is coated with cysteine at the time of planting in combination with the other materials. The cysteine may be in various forms, such as a powder form, a dust form, a slurry form or a liquid form to coat the plant seed.

The present invention also provides a method of accelerating the germination in all plant seeds by applying cysteine to the seed. The cysteine is applied in an amount from 10 mM to 1 nM. In the preferred embodiment, 1 μM or 2.5 g/cwt soybean seed of cysteine is applied.

The present invention also provides a method of enhancing root growth in plants by applying cysteine to the plant. Cysteine is preferably applied in an amount from between about 0.005 to 1.0 lb/A. In the preferred embodiment, 0.01 lb/A of cysteine is applied. The cysteine is applied to enhance root growth by using the aforementioned application methods used for plants and seeds.

A method is also provided for accelerating the onset of flowering in a plant by application of cysteine. The cysteine is applied to the plant in an amount from about 0.005 to 1.0 lb/A as a foliar spray. In the preferred embodiment, 0.01 lb/A of cysteine is applied.

Search for Candidate Auxin-Activated ENOX1 from Plants.

The library of known auxin binding proteins was searched for adenine nucleotide binding sites (GXGXXG), potential protein disulfide interchange sites (CKX), and copper binding sites (H(Y)XH(y)Y)). One such protein containing the appropriate sequence motifs G59LGIAG, C44KK, H106TH and L160LH also containing the auxin binding motif H106THP109GASEVLIVAQ which includes the copper I motif was identified and selected for evaluation as a candidate for the auxin-stimulated ENOX1 from plants (dNOX).

Plasmids construction: Plasmids carrying the open reading frame [M1 to N209 of ABP-20 (Prunus persica)] sequence were prepared by inserting the pET11b vector (between NheI and BamHI sites) with the Arabidopsis ENOX1 sequence. The DNA sequence was synthesized by GenScript USA Inc. (Piscataway, N.J.). DNA sequences of the ligation products (pET11b-ABP-20) were confirmed by DNA sequencing.

Expression of Recombinant dNOX.

The pET11b-ABP-20 was transformed to BL21 (DE3) competent cells. A single colony was picked and inoculated into the 5 ml LB+ampicillin (LB/AMP) medium. The overnight culture (1 ml) was diluted into 100 ml LB/AMP media (1:100 dilution). The cells were grown with vigorous shaking (250 rpm) at 37° C. to an OD₆₀₀ of 0.4-0.6 and IPTG (0.5 mM) was added for induction. Cultures were collected after 16 h incubation with shaking (250 rpm) at 37° C.

Cells were centrifuged at 5,000 g for 6 min. Pellets were then resuspended in 20 mM Tris-HCl, pH 8.0, containing 0.5 mM PMSF, 1 mM benzamidine and 1 mM 6-aminocaproic and lysed by three passages through a French pressure cell (SLM Aminco) at 20,000 psi. Expression of the recombinant ABP-20 of about 20 kDa was confirmed by SDS-PAGE with silver staining. Transformed cells were stored at −80° C. in a standard glycerol stock solution. The recombinant proteins were further purified on Criterion IEF gels (Bio-Rad, Hercules, Calif.). The IEF gel was cut into seven equal segments. The pH represented by each slice was based on IEF standards (Bio-Rad). The slices were soaked in 15 mM Tris-Mes buffer, pH 7, at 4° C. for overnight with shaking. The gel-free extracts were assayed for ENOX activities as follows:

Site-Directed Mutagenesis.

Amino acids indicated were replaced by alanines by site-directed mutagenesis according to Braman et al. (Braman, J., Papworth, C. and Greener, A. 1996. Methods Mol. Biol. 57:32-44). Numbered amino acids and nucleotide positions of splice variant products refer to numbers assigned to amino acids of the full length transcript

### Examples

The identification of the candidate plant, the ENOX1 (YML117W) ENOX1 from Arabidopsis lyrata was based on a homology (BLAST) search by comparison with the ENOX1 (YML117W) from Saccharomyces cerevisiae (FIG. 3). The 14 kDa amino acid sequence selected (FIG. 3) had 37% identity and 58% similarity between amino acids 84 and 126 of XP002882467 from Arabidopsis and amino acids 932-968 of EDN64277 (YML117W) from yeast.

Potential functional motifs within the 14 kDa transcript included a potential NADH binding site at G570XGXXL which aligned with G958XGXXV in YML117W. Potential protein disulfide sites were located at M458XXXXCC and M527XXXXXXC along with C534. Potential copper sites were at H466PY, Y531LY (which over laps M527XXXXXXC) and Y479XXXXH.

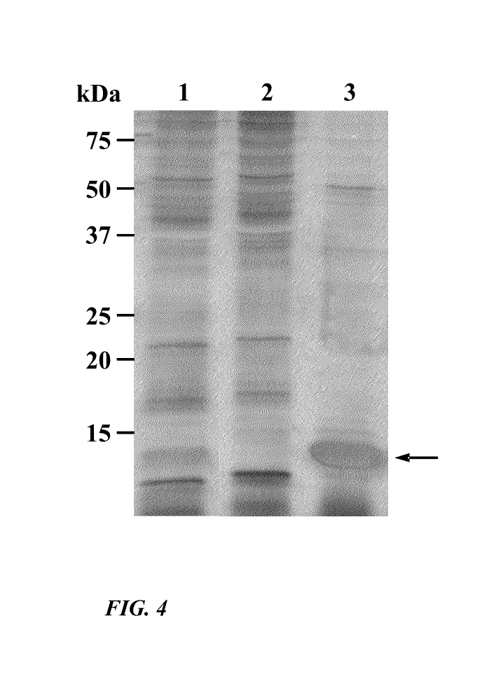

Expression of the recombinant Arabidopsis ENOX1 with a molecular weight of about 14 kDa was confirmed by SDS-PAGE with silver staining (FIG. 4).

Protein Characterization.

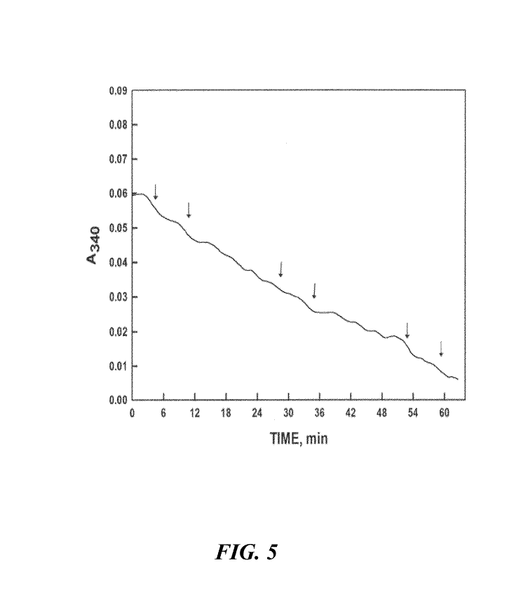

A continuous trace of an IEF-purified preparation of recombinant MBP-tagged cENOX2 illustrates the oscillatory activity characteristic of the ENOX proteins (FIG. 5). Intervals of rapid activity (arrows) were interspersed with intervals of less activity. The period length was 24 min. No oscillations were observed with NADH alone or with the plant ENOX1 in the absence of NADH.

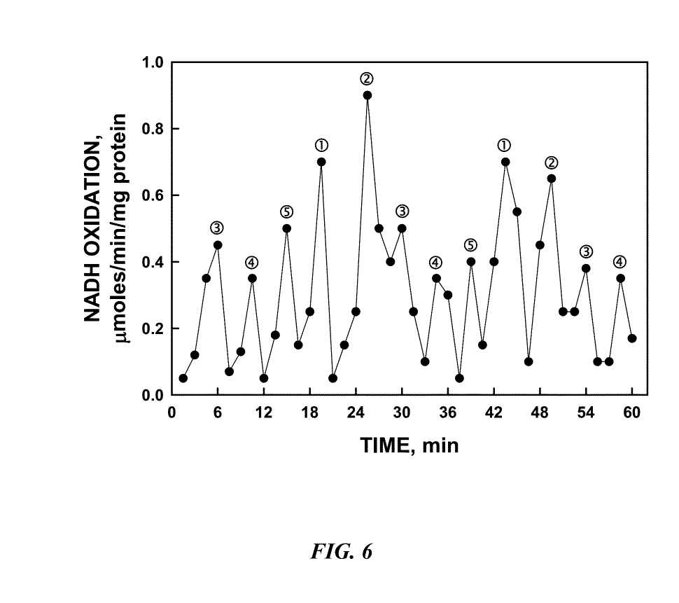

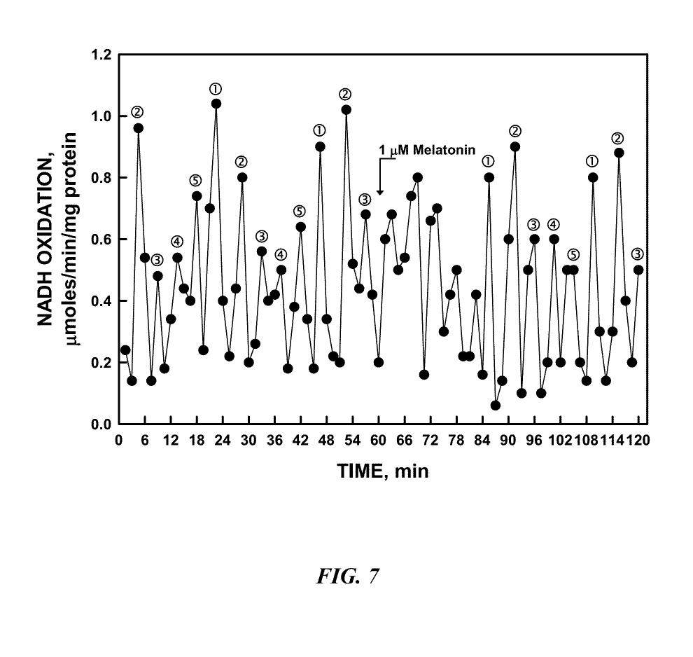

For more detailed evaluations, rates averaged over 1 min every 1.5 min with recombinant plant ENOX1 expressed in bacteria exhibited more clearly the oscillatory pattern of oxidation of exogenously supplied NADH characteristic of ENOX1 proteins (FIG. 6). The repeating pattern was that of five maxima, two of which were separated by six min (arrows) and the remainder separated by 4.5 min [6+(4×4.5)=24 min]. As is characteristic of ENOX1 proteins from other sources, the oscillatory pattern was phased by the addition of 1 μM melatonin (FIG. 7). A new maximum was observed exactly 24 min after melatonin addition and continued thereafter as phased by the melatonin addition.

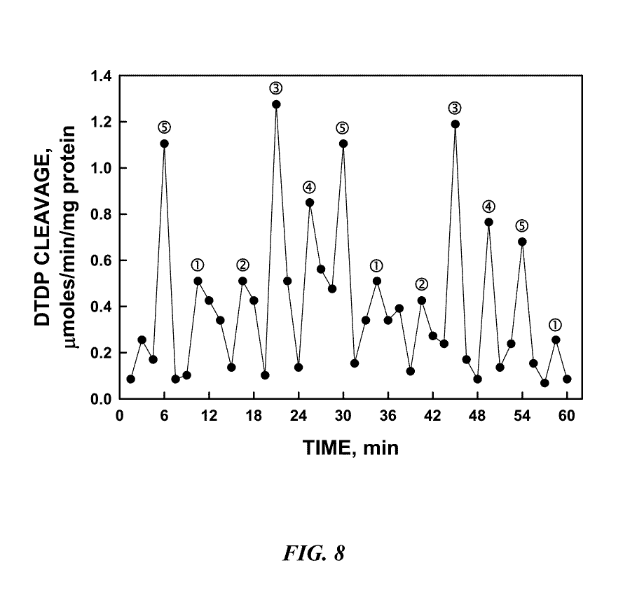

As is characteristic of ENOX proteins in general, the proteins also exhibited protein disulfide-thiol interchange (protein disulfide isomerase) activity illustrated by the time-dependent cleavage of a dithiodipyridyl substrate (FIG. 8). An oscillatory pattern similar to that for NADH oxidation was observed with a period length of 24 min (arrows). The principal maxima of the two activities, NADH oxidation and protein disulfide interchange, alternated.

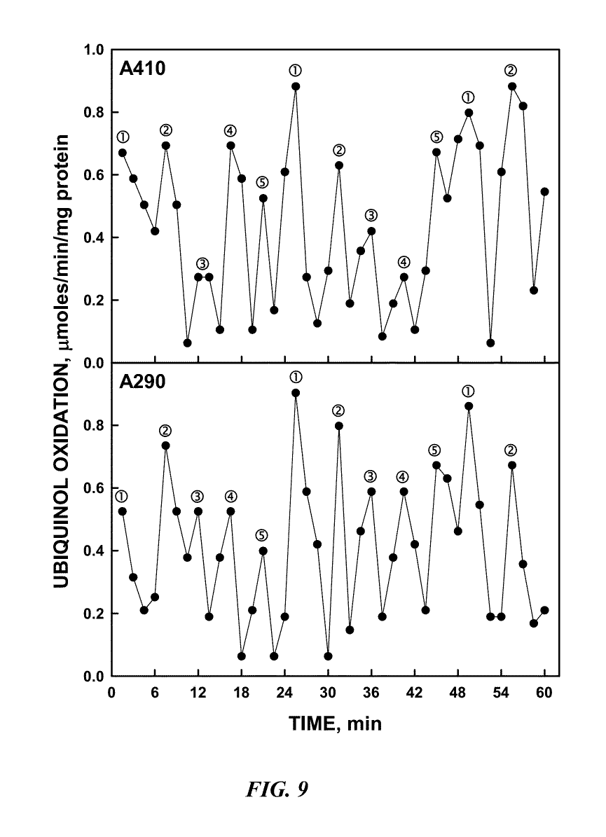

The recombinant ENOX1 oxidizes reduced coenzyme Q in a standard assay (FIG. 9) with activity measured either at A₄₁₀ (FIG. 9A) or at A₂₉₀ (FIG. 9B). as with NADH oxidation (FIG. 6) and dithiodipyridine cleavage (FIG. 8, the characteristic pattern of oscillations with a 24 min period (arrows) was reproduced (FIG. 9). Hydroquinones of the plasma membrane (reduced coenzyme Q for animals/reduced coenzyme Q or phylloquinone for plants) are the physiological substrates for ENOX proteins.

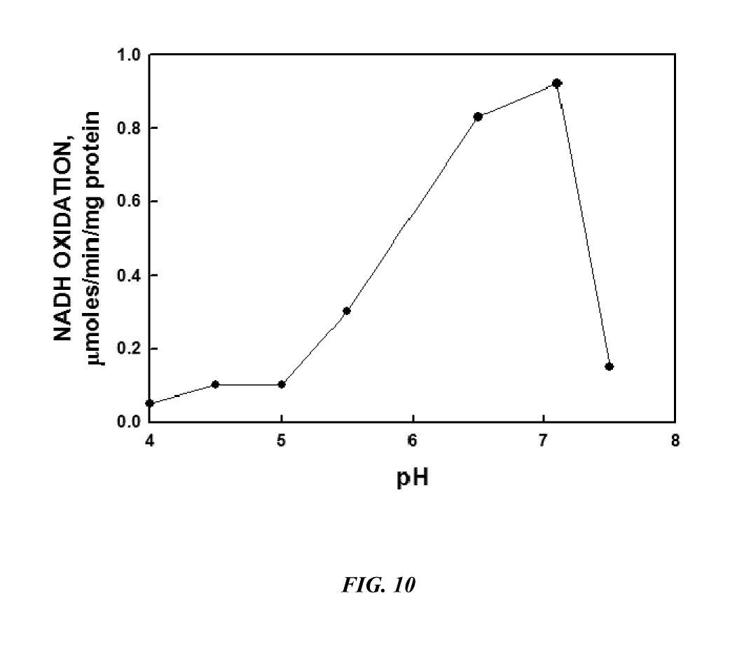

Primarily through reduction of the aggregation of the recombinant proteins, further purification by isoelectric focusing was required to achieve the reported specific activities. Highest specific activities were achieved at a focusing pH of about 6.9 (FIG. 10) which approximates the calculated isoelectric point of the recombinant protein.

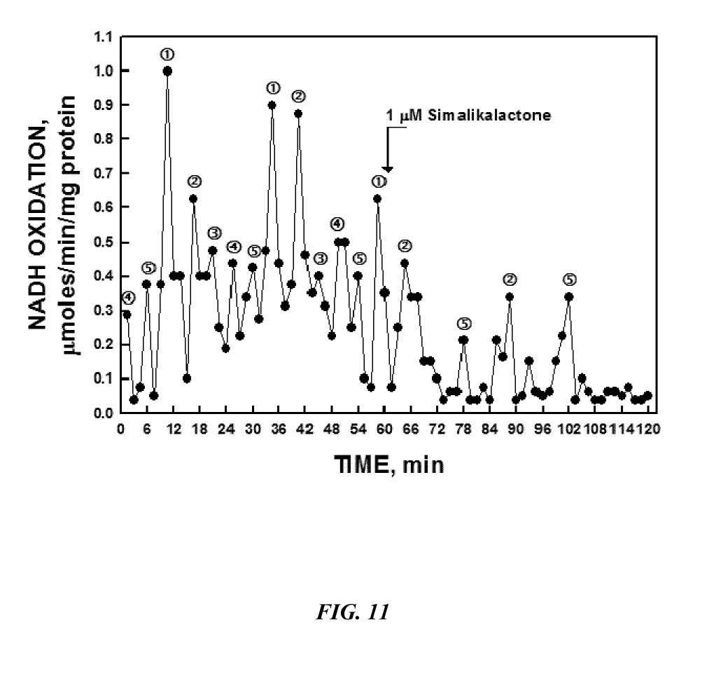

The ENOX activity eluted from the IEF gel was further identified as ENOX1 by its resistance to various ENOX2 inhibitors including cis-platinum, phenoxodiol, EGCg and capsaicin all tested at concentrations sufficient to inhibit ENOX2 activity completely (Table 5). With the recombinant Arabidopsis ENOX1 protein eluted ENOX from the IEF gels, no inhibition was observed. Activity was inhibited by the ENOX1-specific quassinoid inhibitor simalikalactone D (FIG. 11) along with the growth regulating herbicies mefluidide and sulfosulfuron (Table 5). The auxin herbicide 2,4-dichlorophenoxyacetic acid (2,4-D) which stimulates the NOX activity of soybean plasma membranes approximately two-fold at 1 μM, was without effect (Table 5).

dNOX Activity Requires the Presence of Copper.

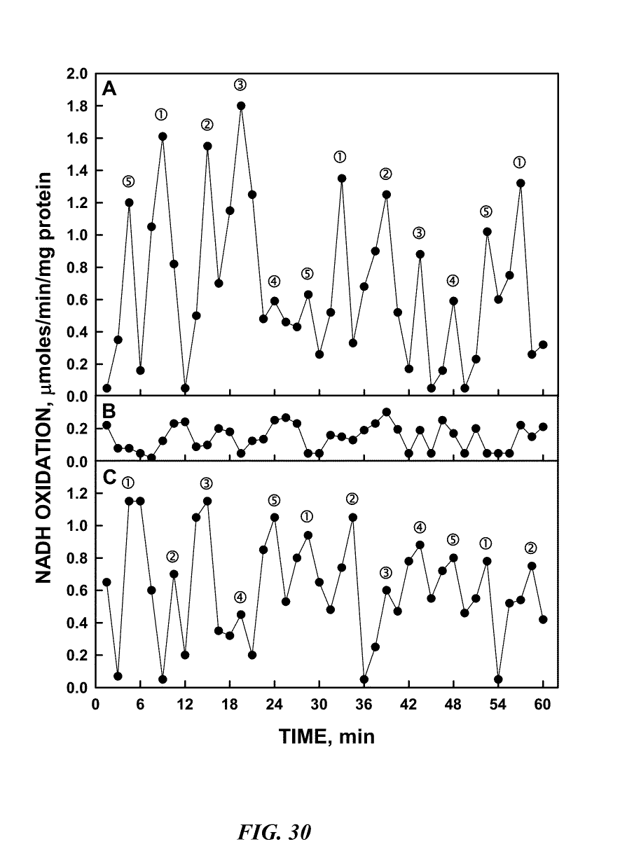

Copper was necessary for dNOX activity (FIG. 30). The IEF-purified dNOX, when unfolded in the presence of trifluoroacetic acid, retained activity after dialysis and at physiological pH (FIG. 30A). However, if the dNOX was unfolded in the presence of trifluoroacetic acid plus the copper chelator bathocuproine, activity was lost (FIG. 30B). Activity was subsequently restored by dialysis to remove the bathocuproine and refolding in the presence of copper at physiological pH (FIG. 30C).

Period Length in Deuterium Oxide.

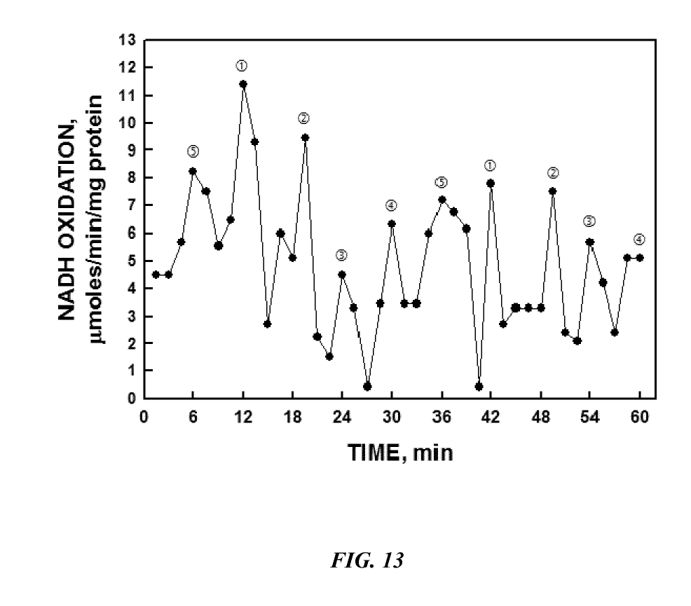

ENOX1 activity when assayed in heavy water yielded a pattern of activities with the period length increased from 24 min to about 30 min (FIG. 13).

Stimulation of NADH Oxidation by Cysteine.

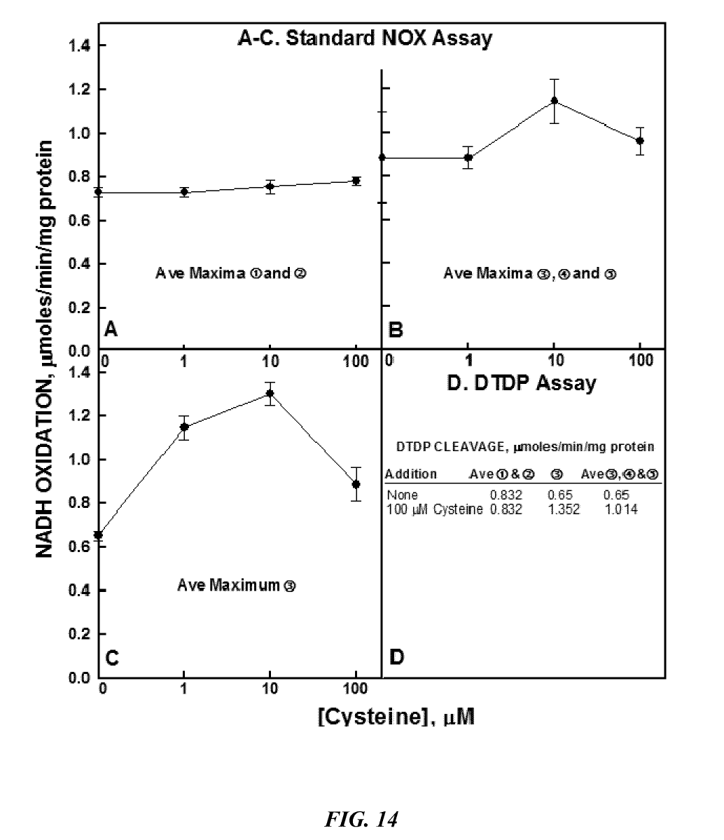

Stimulation of NADH oxidation by cysteine was specific for maximum \textcircled{3} of the ENOX1 activity cycle of recombinant Arabidopsis ENOX1 protein expressed in bacteria (FIG. 14)

TABLE 5
NADH oxidase activity of IEF-purified ENOX1 recombinant Arabidopsis
and response to ENOX2 inhibitors, 2,4-D and the ENOX1 inhibitor
simalikalactone D. Average of 3 determinations ± standard deviations.

| Inhibitor | μmoles/min/mg |
|---|---|
| None | 2.7 ± 0.4 |
| Cis-platinum (100 μM) | 3.5 ± 0.002 |
| Phenoxodiol (10 μM) | 3.7 ± 0.05 |
| EGCg (500 μM) | 3.9 ± 0.05 |
| Capsaicin (1 μM) | 3.7 ± 0.1 |
| Tyrosol (10 μM) | 3.4 ± 0.2 |
| Gallic acid (100 μM) | 3.0 ± 0.5 |
| Simalikalactone D (1 μM) | 0.9 ± 0.1 |
| 2,4-dichlorophenoxyacetic acid (1 μM) | 3.7 ± 0.1 |
| Mefluidide (N-[2,4-Dimethyl-5- | 1.8 ± 0.15 |
| [[(trifluoromethyl)sulfonyl]amino]phenyl] acetamide) | |
| (100 μM) | |
| Sulfonsulfuron sulfonylurea herbicide | 1.2 ± 0.5 |
| (Trade Name: Outrider) (100 μM) | |

The concentrations of cis-platinum, phenoxodiol, EGCg and capsaicin resulted in >90% inhibition of recombinant human ENOX2 assayed in parallel. Whereas, the concentrations of tyrosol and gallic acid used resulted in >90% inhibition of arNOX (ENOX3), 2,4-D at 1 μM which stimulated dNOX of soybean approximately two-fold was without effect. Simalikalactone D is a general ENOX1 inhibitor.

The Expectation that additive TR-III should enhance growth of soybeans is based on the following two premises:
1. The ENOX1 cell surface and growth-related protein and rate of cell activity of elongation (enlargement) are normally in direct proportion; and
2. TR-III irreversibly activates ENOX1 through a conformational change in the ENOX1 protein.

The seedlings grown from the treated seeds show elevated ENOX1 activity as expected.

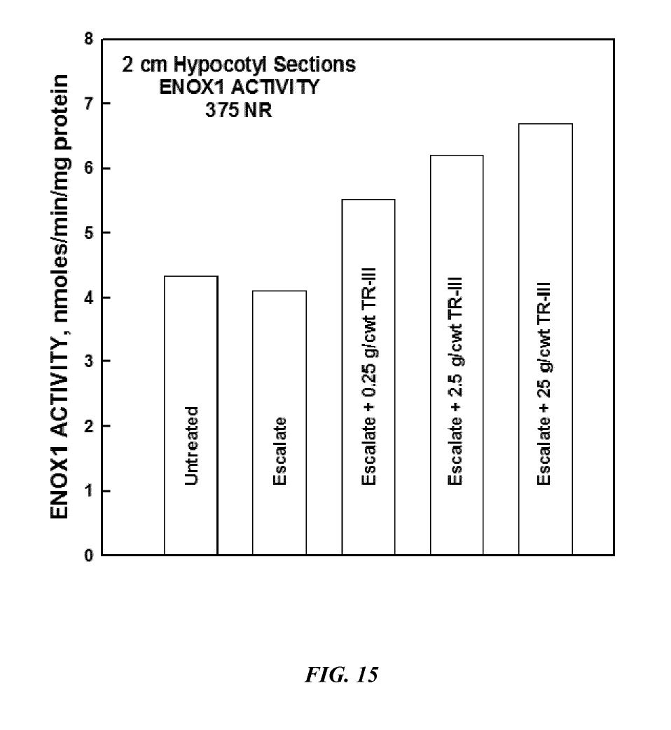

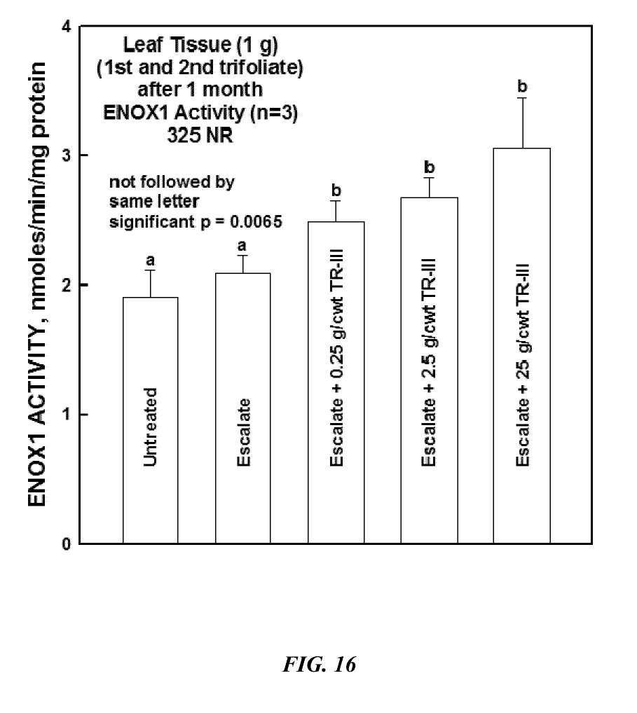

As shown in FIG. 16, the leaves of the plants grown from TR-III-treated seeds showed elevated ENOX1 in roughly the same proportions as for the dark-grown seedlings of FIG. 15.

The irreversible stimulation by TR-III of ENOX1 activity persists as expected and appears to be sustained through a recruitment process.

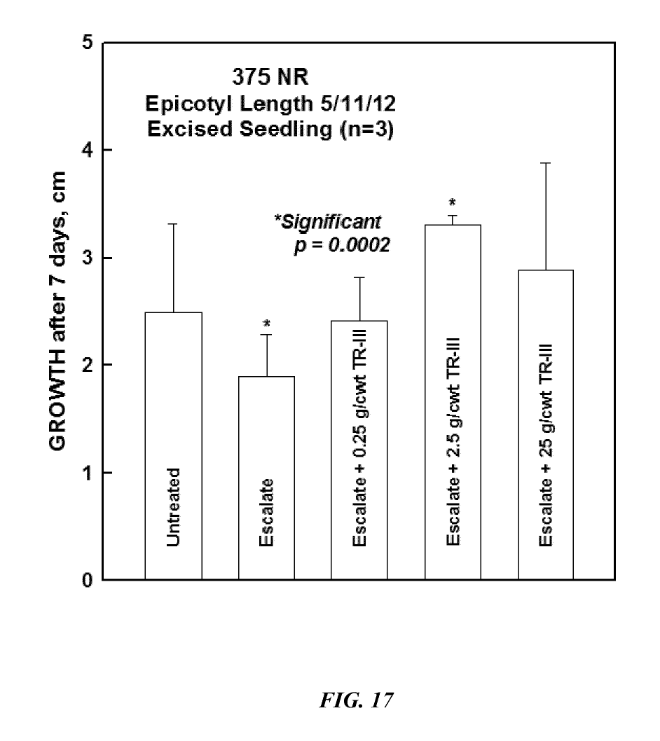

Growth after 1 week was enhanced in seedlings treated with 2.5 g/cwt TR-III compared to Escalate but not for the lower or higher rates (FIG. 17).

Epicotyl elongation was enhanced by seed treatment with Escalate+2.5 g/cwt of TR-III but not in a manner proportional to ENOX1 stimulation of ENOX1 activity as 0.25 or 25 g/cwt had no effect.

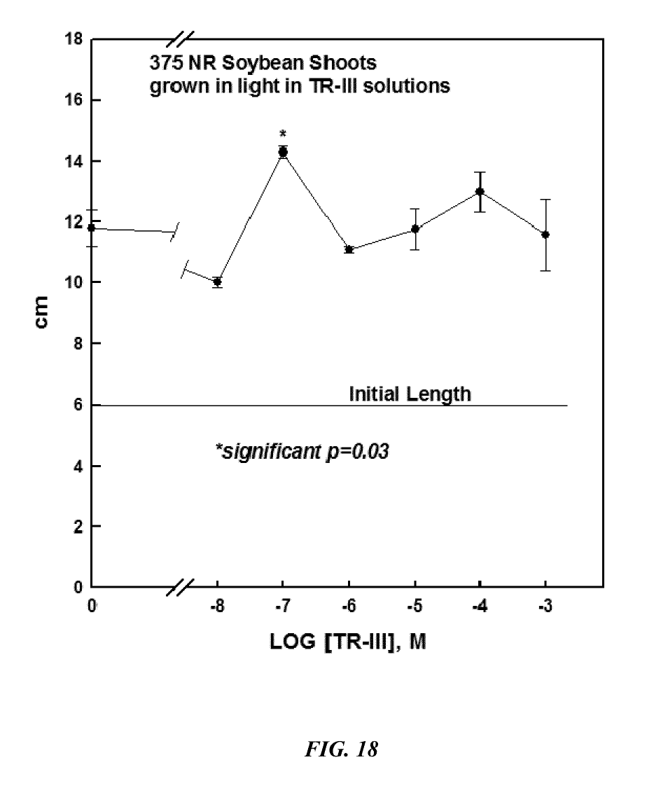

TR-III stimulated shoot growth over a narrow concentration range around 10⁻⁷ M (FIG. 18).

Growth of soybean plants sprayed with TR-III was enhanced by TR-III in greenhouse studies (Table 6).

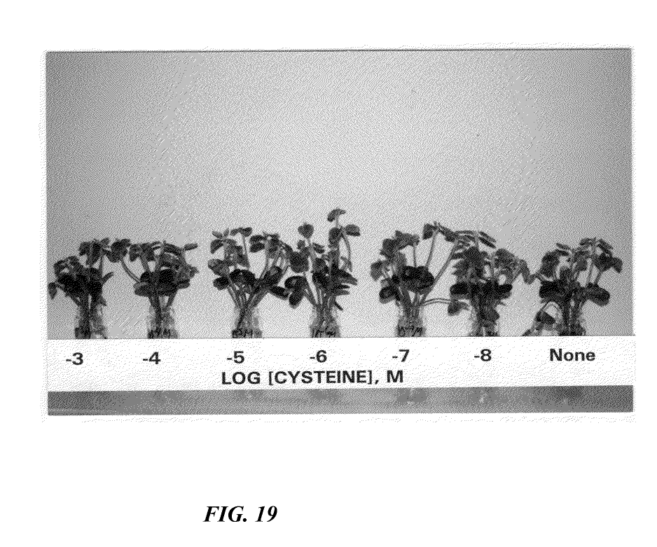

Only with Escalate+0.25 g/cwt TR-III was epicotyl elongation enhanced (50% compared to untreated or Escalate alone) (FIG. 19).

Growth response did not parallel TR-III effects on leaf levels of ENOX1 measured in parallel.

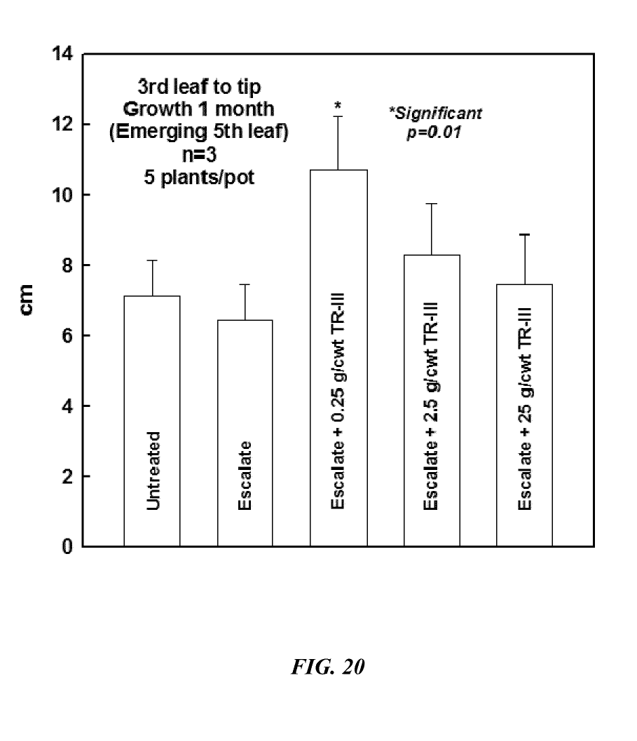

Epicotyl enlargement was observed at 0.01 lb/A. TR-III as in the past with little or no effect from 0.001 or 0.1 lb/A (FIG. 20).

Weight and stem diameter was increased by Escalate plus TR-III at 0.25 and 2.5 g/cwt in greenhouse studies (Table 7).

ENOX1 activity enhanced by TR-III seed treatment was reflected in plasma membranes isolated from 1 cm stem segments of greenhouse grown soybeans (Table 8). For both rates of TR-III there was an increase in about 1 node per plant on average and an increase of 1.2 pods per node to 1.8 pods per node. The number of branches was increased from 0.5 per plant for Escalate alone to 1.5 to 1.6 branches per plant for Escalate+TR-III. Taken together, with an average of 2 pods/branch, 1 extra node and 0.6 extra pods per node, yielded (2+2+6=10) extra pods per plant as observed. With each pod yielding about 0.25 g of beans, this should translate into 10×0.5 g=2.5 extra grams of beans per plant to bring the yield of the TR-III plots to 2.8+2.5=5.3 grams per plant compared to 2.8 g per plant for Escalate alone (Table 9). The principal difference between the low and high rate of TR-III was variability. With 0.25 g/cwt TR-III, important parameters were only marginally significant compared to Escalate alone. However, with 2.5 g/cwt TR-III differences were extremely significant from Escalate alone because of the remarkable agreement among replicates for both Escalate alone and the TR-III plus 2.5 g/cwt Escalate.

Double crop soybean plants harvested on Nov. 5, 2012 responded to TR-III seed treatment by increased pods per plant, increased nodes/plant, branches/plant and stem diameter (Table 9). Plant height was unaffected.

A major indicator that the double crop beans responding to the TR-III, was the increase in stem diameter at the base of the plant just below the first node (Table 9). Stem thickening was confirmed by histological analyses (Table 10). With samples collected in early September, Escalate+2.5 g/cwt TR-III had 3.3+/−0.4 mm of xylem compared to 2.7+/−0.3 mm of xylem for Escalate alone (p=0.0847) which translated into a volume increase in xylem of about 40%. At harvest, the amount of xylem was 5.3 mm for Escalate+2.5 g/cwt of TR-III compared to 4.2 mm of xylem for Escalate alone.

There was an increase in yield of 23% for Escalate+TR-III at 2.5 g/cwt of the double crop soybeans when corrected for stand count.

As compared to a 70% increase in ENOX1 activity of plasma membranes from 1 cm stem segments harvested between the second and third trifoliate leaf in the greenhouse (Table 11).

TABLE 6
TR-III spray. Greenhouse grown 375 NR soybeans sprayed 14 days
after planting and measured 10 days after spraying.

| | Plant height above cotyledons | | | |
|---|---|---|---|---|
| Experiment Number | 0 | 0.001 lb/A | 0.01 lb/A | 0.1 lb/A |
| I | 13.3 | 14.8 | 14.3 | 14.4 |
| II | 13.9 | 14.6 | 14.5 | 15.9 |
| Ave + MAD | 13.6 ± 0.3 | 14.7 ± 0.1 | 14.4 ± 0.1 | 15.2 ± 0.7 |

TABLE 7
Weight and stem diameter of 1st internode above the cotyledons.
Average of 3 replications of 5 plants each.
Greenhouse grown 375 NR soybeans 40 days after planting.

| | Wt/1 cm stem section | |
|---|---|---|
| | (g) | Stem diameter |
| Untreated | 0.31 ± 0.04 | 4.3 ± 0.3 |
| Escalate | 0.32 ± 0.04 | 4.3 ± 0.5 |
| Escalate + | 0.33 ± 0.04 | 4.4 ± 0.05 |
| TR-III 0.25 g/cwt | | |
| Escalate + TR-III 2.5 g/cwt | 0.34 ± 0.03 (8%) NS | 5.1 ± 0.04 (19%)* |
| Escalate + TR-III 25 g/cwt | 0.35 ± 0.03 (11%) | 4.75 ± 0.35 |

*Significant p = 0.05

TABLE 8
ENOX1 activity of plasma membranes from 2 cm stem segments
harvested between the second and third trifoliate leaf of
greenhouse grown 375 NR soybeans 40 days after planting.
Duplicate determinations from 3 replicates of 5 plants each.

| Treatment | μmoles/min/mg protein |
|---|---|
| None | 0.34 ± 0.04 |
| Escalate | 0.35 ± 0.04 |
| Escalate + 0.25 g/cwt TR-III | 0.34 ± 0.05 |
| Escalate + 2.5 g/cwt TR-III | 0.60 ± 0.05 |
| Escalate + 25 g/cwt TR-111 | 0.46 ± 0.01 |

TABLE 9
Summary of 2012 Arcadia South DC Soybean Treatment Study.
Beck Plots, Atlanta, IN.

| | | Treatment | |
|---|---|---|---|
| | | Escalate + | Escalate + |
| | Escalate | TR-III 0.25 g/cwt | TR-III 2.5 g/cwt |
| Height (in) | 23 ± 1 | 23 ± 3* | 25 ± 1* |
| Pods/plant | 11.7 ± 0.5 | 20.0 ± 6.7** | 21.0 ± 1.0**** |
| Nodes/plant | 9.8 ± 0.5 | 10.9 ± 1.8* | 11.7 ± 0.5*** |
| Branches/plant | 0.5 ± 0.2 | 1.5 ± 0.9* | 1.6 ± 0.2*** |
| Empty pods/plant | 0.5 | 0.7 | 1.2 |
| (Average) | | | |
| Stem diam (cm) | 0.4 ± 0.5 | 0.51 ± 0.08* | 0.52 ± 0.03*** |
| Pods/node | 1.2 | 1.8 | 1.8 |
| (Average) | | | |
| Seed wt/plant (g) | 2.8 ± 0.35 | 5.2 ± 1.9** | 5.5 ± 0.3**** |
| Yield bu/A | 28 | 32 | 34 |

Planted: Jun. 27, 2012
Tillage: No-Till
Previous Crop: Wheat
Rows: 11
Row Width: 7.5″
Replications: 3
Harvested: Nov. 5, 2012(Average 20 plants/replicate)
*Not significant
**p = 0.075-0.098 (marginally significant)
***p = 0.0025-0.0039 (very significant)
****p = 0.0001-0.0005 (extremely significant

TABLE 10
Soybean xylem diameters and area measured histologically
from 10 to 12 sections from 3 plants at maturity.

| Treatment | Diameter (mm) | Area |
|---|---|---|
| Escalate | 1.36 ± 0.16 | 1.4 |
| Escalate + 0.25 g/cwt TR-III | 1.35 ± 0.22 | 1.4 |
| Escalate + 2.5 g/cwt TR-III | 1.61 ± 0.17* | 2 |

*Significant p = 0.0847. Equivalent to a 40% increase in xylem surface area.

TABLE 11
ENOX1 activity of plasma membranes from 1 cm stem segments
harvested between the second and third trifoliate leaf of
Becks 375 NR greenhouse grown soybeans.
Duplicate determination comparing averages ± standard deviations
from 3 pots of 5 plants per pot assayed each plant.

| Treatment | μmoles/min/mg protein |
|---|---|
| None | 0.34 ± 0.04 |
| Escalate | 0.35 ± 0.04 |
| Escalate + 0.25 g/cwt TR-III | 0.34 ± 0.05 |
| Escalate + 2.5 g/cwt TR-III | 0.60 ± 0.05* |
| Escalate + 25 g/cwt TR-III | 0.46 ± 0.01** |

*Very significant (p = 0.002)
**Very significant (p = 0.007)

TABLE 12
Growth and plasma membrane ENOX1 activity of transgenic ST109-2-4
(10 plants) and Williams 82 ISU (20 plants) soybeans grown in the
greenhouse 2 months after planting.

| | Plant | Stem | ENOX1, μmoles/min/mg protein | |
|---|---|---|---|---|
| | Height | Diameter | | +100 μM |
| Seed Source | (cm) | (mm) | −cysteine | cysteine |
| ST-109-2-4 | 51 ± 5* | 4.2 ± 0.2** | 0.110 ± 0.004 | 0.123 ± 0.005* |
| Williams 82 | 44 ± 2 | 4.0 ± 0.2 | 0.060 ± 0.003 | 0.072 ± 0.003 |
| ISU | | | | |

Williams 82 ISU = Williams 82 ISU GH 2010 row 1 + row 2 and row 2B1
ENOX1 activities were measured on plasma membranes prepared from the emerging trifoliate leaf and stem harvested 1 cm below the emerging trifoliate leaf. Trifoliate leaf and stem tissues were note different and reported values are averages of both ± standard deviations.
*Significantly different from Williams 82 ISU p < 0.001
**Significantly different from Williams 82 ISU p = 0.015

ST-109-2-4 soybean plants harvested 2 months after planting in the greenhouse exhibited 80% elevated activities of ENOX1 associated with plasma membranes isolated from emerging trifoliate leaves and stem segments harvested just below the emerging trifoliate compared to Williams 82. The plants, however, were only 16% taller ad basal stem diameters were increased only 5%. The plasma membrane ENOX1 activity of both the transgenic ST-109-2-4 and the Williams 82 plants responded to added 100 μM cysteine by about 12%. These data demonstrate that overexpressed ENOX1 in the transgenic plants reaches the plasma membrane and is still responsive to added cysteine but the growth response is disproportionately less.

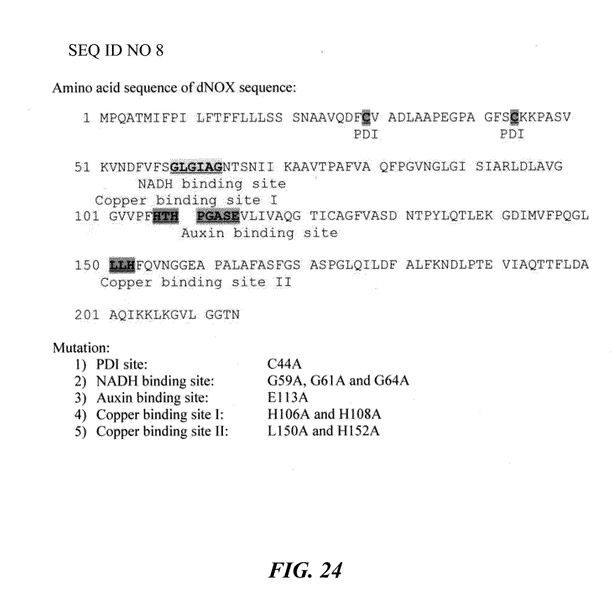

The identification of the candidate plant auxin-activated ENOX protein (dNOX) was based on a homology search of known auxin-binding proteins that also contained the corresponding functional motifs of known ENOX proteins. The 20 kDa amino acid sequence selected, ABP-20 (FIG. 24, SEQ ID NO: 8), contained the required functional motifs within the 20 kDa transcript that included a potential NADH binding site at G59LGTAG, a potential protein disulfide site located at C44KK and along potential copper sites were at H106TH and L160LH along with the auxin binding motif H106THPGASSVLIVAQ.

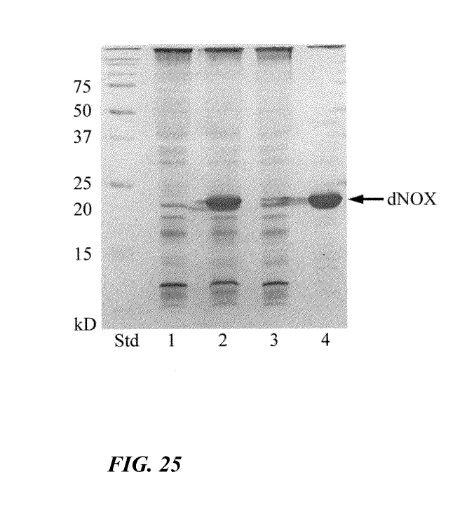

Expression of the recombinant ABP-20 with a molecular weight of about 20 kDa was confirmed by SDS-PAGE with silver staining (FIG. 25).

Protein Characterization.

At no point during the purification did the recombinant protein exhibit NADH oxidase activity above the background rate of NADH auto-oxidation in the absence of auxin addition. Upon addition of auxin (e.g., 1 μM 2,4-D) the activity was enhanced 10 to 20 fold above base line activity with an average specific activity of ca 0.6±0.2 μmoles/min/mg protein with IEF-purified fractions.

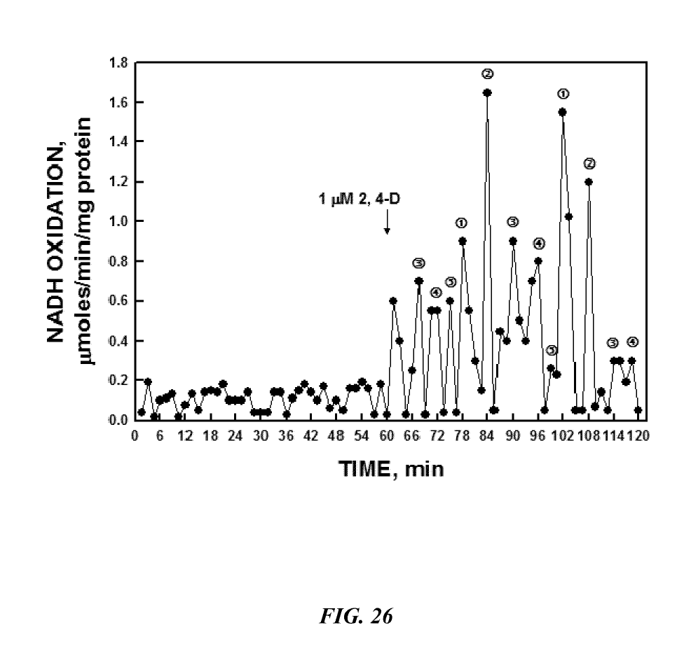

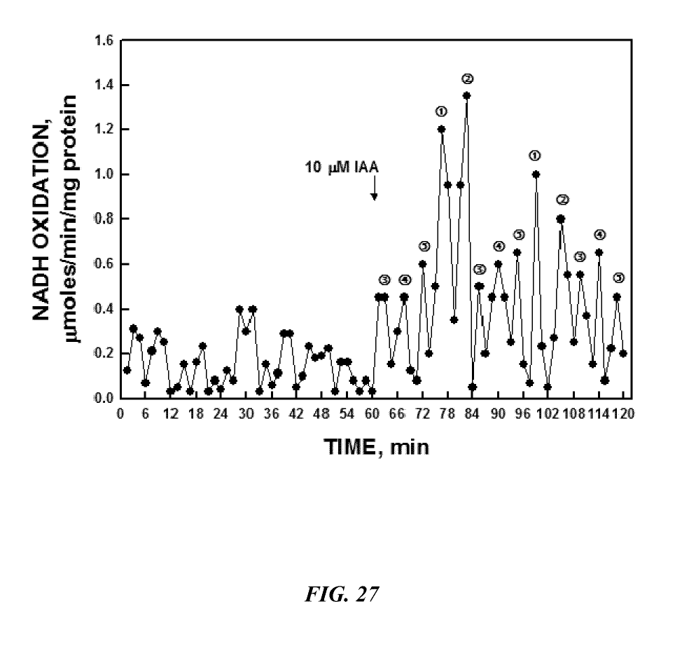

For more detailed evaluations, rates averaged over 1 min every 1.5 min with recombinant plant ENOX1 expressed in bacteria and purified by isoelectric focusing exhibited clearly the oscillatory pattern of oxidation of exogenously supplied NADH characteristic of ENOX1 proteins (FIG. 26). The repeating pattern was that of five maxima, two of which were separated by 6 min (maxima \textcircled{1} and \textcircled{2}) and the remainder (maxima \textcircled{3}, \textcircled{4} and \textcircled{5}) separated by 4.5 min [6+(4×4.5 min)=24 min]. As is characteristic of ENOX1 proteins from other sources, the maxima labeled \textcircled{1} and \textcircled{2} were more prominent than the maxima \textcircled{3}, \textcircled{4} and \textcircled{5}. Similar results were obtained when the natural auxin, indole-3-acetic acid (IAA), was substituted for the 2,4-D (FIG. 27).

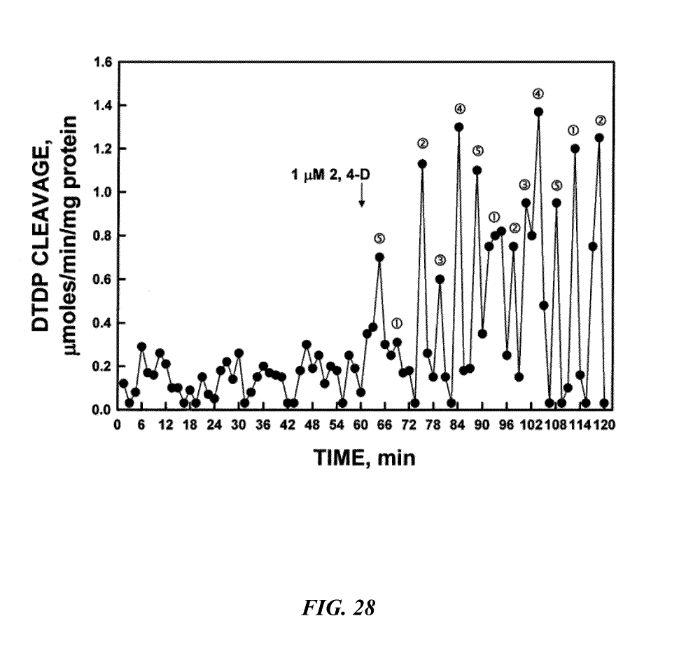

As is characteristic of ENOX proteins in general, the proteins also exhibited protein disulfide-thiol interchange (protein disulfide isomerase) activity illustrated by the time-dependent cleavage of a dithiodipyridyl substrate (FIG. 28). An oscillatory pattern similar to that for NADH oxidation was observed with a period length of 24 min. As reported previously (Morré, D. J. and Morré, D. M. 2003. Free Radical Res. 37: 795-808), with DTDP the maxima labeled \textcircled{3}, \textcircled{4} and \textcircled{5} were more pronounced than those labeled \textcircled{1} and \textcircled{2} suggesting an alternation of the principal maxima of NADH oxidation and protein disulfide interchange.

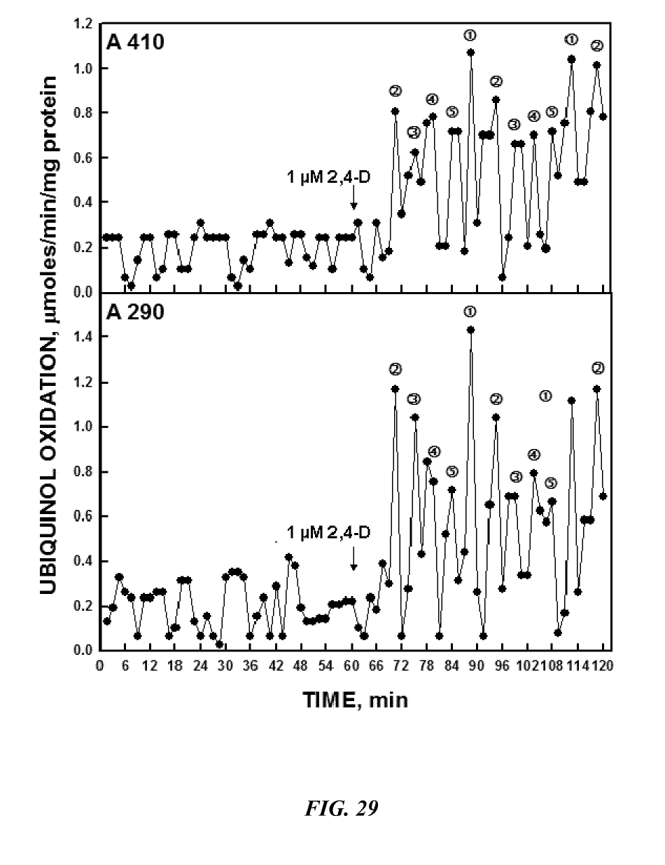

The recombinant ENOX1 oxidizes reduced coenzyme Q in a standard assay (FIG. 29) with activity measured either at A₄₁₀ (FIG. 29A) or at A₂₉₀ (FIG. 29B). As with NADH oxidation (FIG. 27) maxima labeled \textcircled{1} and \textcircled{2} were more pronounced than those labeled \textcircled{3}, \textcircled{4} and \textcircled{5}. Hydroquinones of the plasma membrane (reduced coenzyme Q for animals/reduced coenzyme Q or phylloquinone for plants) are the physiological substrates for ENOX proteins.

Primarily through reduction of the aggregation of the recombinant proteins, further purification by isoelectric focusing was required to achieve the reported specific activities. Highest specific activities were achieved at a focusing pH of about 5.0 which approximates the calculated isoelectric point of the recombinant protein of pH 5.19.

Activity was inhibited by the thiol reagents PCMB and PCMS (Table 12). The inactive auxin analog 2,3-dichlorophenoxyacetic acid (2,3-D) was without effect as was the ENOX1-specific quassinoid inhibitor simalikalactone D (Table 12). The anticancer drugs cis platinum, doxorubicin (Adriamycin) and ENOX2 specific quassinoid inhibitor glaucarubolone, which inhibit auxin-induced growth but not control growth in plants (Morré, D. J., Crane, F. L., Barr, R., Penel, C. and Wu, L. Y. 1988. Physiol. Plant. 72: 236-240), also inhibited the activity of the recombinant protein. The growth inactive transplatinum was without effect (Table 12).

ENOX1 Activity Requires the Presence of Copper.

Copper was necessary for ENOX1 activity (FIG. 30). The IEF-purified ENOX1, when unfolded in the presence of trifluoroacetic acid, retained activity after dialysis and at physiological pH (FIG. 30A). However, if the ENOX1 was unfolded in the presence of trifluoroacetic acid plus the copper chelator bathocuproine, activity was lost (FIG. 30B). Activity was subsequently restored by dialysis to remove the bathocuproine and refolding in the presence of copper at physiological pH (FIG. 30C).

Confirmation of Functional Assignments of ABP-20 Motifs by Site-Directed Mutagenesis.

Confirmation of functional assignments of motifs common to ENOX proteins is provided for the specific functional motifs of dNOX (ABP-20) by site directed mutagenesis (Table 13). Within the CKK motif common to ENOX1 proteins, activity was reduced by 81% in the C44A replacement for both NADH oxidation and protein disulfide-dithiol interchange activity. The G59A replacement in the putative adenine nucleotide binding motif largely eliminated NADH oxidation and was without effect on disulfide-thiol interchange. The E113H replacement in the auxin binding motif also eliminated the auxin-stimulation of NADH oxidase activity. Putative copper site replacements, H106A and H152A, reduced activities of both NADH oxidation and disulfide-thiol interchange to near background.

TABLE 13
NADH oxidase activity of IEF-purified recombinant ABP-20 and
response to auxins and ENOX inhibitors.
Average of 3 determinations ± standard deviations.

| Addition | Concentration | μmoles/min/mg |
|---|---|---|
| None | | 0.1 ± 0.08 |
| 2,4-dichlorophenoxyacetic acid (2,4-D) | 1 μM | 0.8 ± 0.2 |
| 2,3-dichlorophenoxyacetic acid (2,3-D) | 1 μM | 0.15 ± 0.01 |
| Indole-3-acetic acid (IAA) | 1 μM | 0.8 ± 0.05 |
| PCMB | 100 μM | 0.1 ± 0.05 |
| PCMS | 100 μM | 0.3 ± 0.2 |
| Cis-platinum | 1 μM | 0.2 ± 0.05 |
| Trans-platinum | 1 μM | 0.7 ± 0.1 |
| Doxorubicin (Adriamycin) | 1 μM | 0.2 ± 0.05 |
| Simalikalactone D | 1 μM | 0.65 ± 0.07 |
| Glaucarubolone | 1 μM | 0.2 ± 0.06 |

TABLE 14
Confirmation of functional motifs of dNOX (ABP-20) by site-directed
mutagenesis.

μmoles/min/mg protein

| | | DTDP |
|---|---|---|
| Modification | NADH Oxidation | Interchange |
| None (Wild Type) | 0.8 ± 0.1 | 0.9 ± 0.05 |
| C44A | 0.15 ± 0.05 | 0.02 ± 0.01 |
| G59A | 0.06 ± 0.02 | 0.07 ± 0.02 |
| E113A | 0.03 ± 0.01 | 0.04 ± 0.02 |
| H106A | 0.04 ± 0.01 | 0.02 ± 0.01 |
| H152A | 0.03 ± 0.01 | 0.02 ± 0.01 |

The uses of the terms “a” and “an” and “the” and similar references in the context of describing the invention, especially in the context of the following claims, are to be construed to cover both the singular and the plural unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

While the invention has been illustrated and described in detail in the drawings and the foregoing description, the same is to be considered as illustrative and not restrictive in character, it being understood that only the preferred embodiment has been shown and described and that all changes and modifications that come within the spirit of the invention are desired to be protected. In addition, all references cited herein are indicative of the level of skill in the art and are hereby incorporated by reference in their entirety.

What is claimed is:

1. A DNA construct comprising an isolated DNA that encodes for an ecto-nicotinamide dinucleotide oxidase disulfide thiol exchange protein.

2. The DNA construct of claim 1, wherein said DNA construct is a plasmid.

3. The DNA construct of claim 2, wherein said DNA construct is a pET11a vector.

4. The DNA construct of claim 3, wherein said DNA sequence is located between the NheI and BamHI sites of a pET11a vector.

5. The DNA construct of claim 1, wherein said ecto-nicotinamide dinucleotide oxidase disulfide thiol exchange protein is a recombinant oxidase disulfide thiol exchange protein.

6. The DNA construct of claim 1, wherein said ecto-nicotinamide dinucleotide oxidase disulfide thiol exchange protein is a mammalian oxidase from Homo sapiens.

7. The DNA construct of claim 1, wherein said ecto-nicotinamide dinucleotide oxidase disulfide thiol exchange protein is a fission yeast from Saccharomyces cerevisiae.

8. The DNA construct of claim 1, wherein said ecto-nicotinamide dinucleotide oxidase disulfide thiol exchange protein is a higher plant oxidase from the genus Arabidopsis.

9. The DNA construct of claim 1, wherein said ecto-Nicotinamide dinucleotide oxidase thiol interchange protein is a higher plant oxidase from the genus Prunus.

10. The DNA construct of claim 1, wherein said DNA sequence is SEQ ID NO: 1.

11. The DNA construct of claim 1, wherein said DNA sequence is SEQ ID NO: 2.

12. The construct of claim 1, wherein said DNA sequence is SEQ ID NO: 3.

13. The construct of claim 1, wherein said DNA sequence is SEQ ID NO: 4.

14. A bacterial cell comprising the construct of claim 1.

15. The bacterial cell of claim 14, wherein said bacterial cell is of the species Agrobacterium tumefaciens.

16. A chimeric gene capable of expressing a polypeptide in a plant comprising a DNA encoding for the polypeptide wherein said polypeptide is an ecto-nicotinamide dinucleotide oxidase disulfide thiol exchange protein.

17. The gene of claim 16, wherein said DNA encodes for an ecto-nicotinamide dinucleotide oxidase disulfide thiol exchange protein from Homo sapiens.

18. The gene of claim 16, wherein said DNA encodes for an ecto-nicotinamide dinucleotide oxidase disulfide thiol exchange protein from the genus Arabidopsis.

19. The gene of claim 16, wherein said DNA encodes for an ecto-nicotinamide dinucleotide oxidase disulfide thiol exchange protein from a Saccharomyces cerevisiae.

20. A microorganism containing the chimeric gene of one of claim 16.

21. A plant containing the chimeric gene of claim 16.

22. A plant seed containing the chimeric gene of claim 16.

23. The plant of claim 21, wherein said plant is a soybean, maize, sorghum, vegetable, root crop, fruit, or forage plant.

24. The plant seed of claim 22, wherein said plant seed is a soybean seed, maize seed, sorghum seed, vegetable seed, root crop tuber, fruit seed, or forage plant seed.

25. A method for increasing the activity of an ecto-nicotinamide dinucleotide oxidase disulfide thiol exchange protein in a plant containing the chimeric gene of claim 16, comprising adding an ENOX activator to the plant.

26. The method of claim 25, wherein said ENOX activator is cysteine.

27. The method of claim 25, wherein said ENOX activator is an auxin.

28. A seed coating for a transgenic plant seed containing the chimeric gene of claim 16 comprising an ENOX activator.

29. The seed coating of claim 28, wherein said ENOX activator is cysteine.

30. A method for cultivating a plant containing the chimeric gene of claim 16, comprising spraying a composition comprising cysteine as a foliar spray.

31. A method for inducing early flowering to a crop of soybeans comprising the chimeric gene of claim 16, comprising administering an auxin or ENOX activator to said crop.

32. The method of claim 31, wherein said ENOX activator is cysteine.
